(19) United States
(12) Patent Application Publication
(10) Pub. No.: US 2025/0066812 A1
(43) Pub. Date: Feb. 27, 2025

(54) RECOMBINANT AAV FOR THE GENE THERAPY OF FABRY DISEASE

(71) Applicant: Skyline Therapeutics Limited, Cayman Islands (GB)

(72) Inventors: Jinzhao HOU, Shanghai (CN); Ting YU, Shanghai (CN); Jiao YUE, Shanghai (CN); Haiyan JIANG, Shanghai (CN)

(73) Assignee: Skyline Therapeutics Limited, Cayman Islands (GB)

(21) Appl. No.: 18/725,000

(22) PCT Filed: Dec. 30, 2022
(86) PCT No.: PCT/CN2022/144064

§ 371 Date: Jun. 27, 2024

(30) Foreign Application Priority Data
Dec. 31, 2021 (WO) PCT/CN2021/143573

(51) Int. Cl. C12N 15/86; A61K 35/761; A61K 38/47; A61K 48/00; C12N 9/40

(52) U.S. Cl. CPC C12N 15/86; A61K 35/761; A61K 38/47; A61K 48/0041; C12N 9/2465; C12N 2750/14143; C12N 2840/60; C12Y 302/01022

(57) ABSTRACT

The present invention provides a polynucleotide and recombinant AAV encoding human alpha galactosidase A. The present invention also provides a method of treating Fabry disease comprising administering the recombinant AAV to a subject in need thereof.

### TECHNICAL FIELD

The present invention is related to gene therapy. In particular, the present invention involves the recombinant adeno-associated virus (AAV) for the gene therapy of Fabry disease.

### BACKGROUND

Fabry disease is an X-linked lysosomal storage disease caused by loss of function mutations in the GLA gene encoding alpha galactosidase A (α-Gal A)[1]. Deficiency of α-Gal A leads to accumulation of its substrates globotriaosylceramide (Gb3) and globotriaosylsphingosine (lyso-Gb3) in various cells including vascular cells (endothelial and smooth muscle cells), cardiac cells (endocardial cells, cardiomyocytes, and cells in cardiac valves), kidney epithelial cells (podocytes, tubular and glomerular cells), and neurons of the peripheral nerves system (dorsal root ganglia)[2]. Male hemizygote patients develop symptoms including extremity neuropathic pain, angiokeratoma, progressive renal insufficiency, cardiac abnormalities and cerebrovascular stroke[1,3]. Female carriers are often affected with more variable and less severe symptoms[4]. Median survival is 50-55 years for male patients and 60-70 years for females[5].

Several therapies have been approved for Fabry disease[2,6]. The first approved therapy is ERT (agalsidase alpha/agalsidase beta), which is able to slow down/stabilize the disease if started early[7,8]. However patients still develop severe clinical events including cardiac death, renal failure and stroke, etc[9]. Further, the bi-weekly intravenous infusion of the recombinant enzyme is a life-long costly treatment. Other approved therapy includes oral small molecules (migalastat) that act as pharmacological chaperone to restore the function of the misfolded α-Gal A protein[10,11]. However, this approach depends on the nature of the GLA mutations; only patients with migalastat-amenable mutant forms of GLA will respond to the therapy, therefore limiting the patient population that might benefit from the therapy[10,11].

The unmet need in the art is to develop novel therapies that could augment cardiac and renal correction and be more convenient to patients.

### SUMMARY OF INVENTION

In order to meet the need, the inventors have developed a gene therapy for Fabry disease which offers a persistent and endogenous production of α-Gal A following the transfer of a functional copy of the GLA gene such as a GLA gene encoding a wild-type GLA polypeptide to a patient.

In a first aspect, the present invention provides a polynucleotide of interest comprising a nucleotide sequence selected from SEQ ID NOs: 1, 2, 3 and 4.

In a second aspect, the present invention provides an expression construct comprising a polynucleotide of interest that comprises a nucleotide sequence selected from SEQ ID NOs: 1, 2, 3 and 4 and is operably linked to a promoter. In some embodiments, the nucleotide sequence is SEQ ID NO: 4.

In some embodiments, the construct further comprises an intron. In some embodiments, the intron is between the promoter and the polynucleotide of interest. In some embodiments, the intron is at least 200 nucleotides in length. In some embodiments, the intron is selected from a chicken beta-actin (CBA) chimeric intron and a modified CBA chimeric intron set forth in SEQ ID NOs: 5 and 6, respectively. In some embodiments, the construct further comprises an enhancer. In some embodiments, the enhancer is upstream of the promoter. In some embodiments, the enhancer is a CMV early enhancer set forth in SEQ ID NO: 9. In some embodiments, the promoter is a chicken beta-actin core promoter set forth in SEQ ID NO: 10.

In a third aspect, the present invention provides a recombinant adeno-associated virus (rAAV) comprising a genome comprising the expression construct of the present invention. In some embodiments, the rAAV is prepared by a system containing a transgene plasmid comprising the genome of the rAAV, a packaging plasmid encoding the REP and/or CAP proteins, and a helper plasmid. Therefore, the present invention further provides a vector comprising the expression construct of the invention.

In a fourth aspect, the present invention provides a pharmaceutical composition comprising the rAAV of the present invention.

In a fifth aspect, the present invention provides a host cell comprising the polynucleotide, the expression construct or the rAAV of the present invention.

In a sixth aspect, the present invention provides a method of treating a disease associated with the deficiency of alpha galactosidase A (α-Gal A), comprising administering the rAAV or the pharmaceutical composition of the present invention to a subject in need thereof.

The present invention also provides use of the polynucleotide, the expression construct, the rAAV, the pharmaceutical composition and/or the host cell of the present invention in the preparation of a medicament for treating a disease associated with the deficiency of α-Gal A in a subject in need thereof.

In some embodiments, the disease is Fabry disease.

### BRIEF DESCRIPTION OF THE DRAWINGS

FIG. 1 shows the structure of the constructs for expressing hGLA (Panel A) and the map of a transgene plasmid (Panel B, the transgene plasmid comprising construct 108 of Example 1).

FIG. 2 shows the GLA activity in the tissues of wild-type mice administered with the rAAVs; the rAAVs are referred to as the construct # for preparing the same, which applies in FIGS. 3-7.

FIG. 3 shows the GLA activity and Gb3 concentration in the plasma of GLA knock-out (KO) mice administered with the rAAVs. Panel A: GLA activity in plasma; Panel B: levels of increased GLA activity were expressed as fold change of the quantity in plasma from age-matched wild-type male mice; Panel C: Gb3 concentration in plasma; and Panel D: percent of Gb3(%) remaining in the plasma relative to the GLA KO male mice treated with DPBS. Error bars indicate standard deviation. “HE” represents one of the GLA copies was mutated, “HO” represents homozygous knockout mouse, the same applies to the following Figures, and “KO” is generally identical to “HO”.

FIG. 4 shows the GLA activity and Gb3 concentration in the kidney of GLA KO mice administered with the rAAVs. Panel A: GLA enzyme activity in kidney; Panel B: levels of increased GLA enzyme activity were expressed as fold change of the quantity in kidney from age-matched wild-type male mice; Panel C: Gb3 concentration in kidney; and Panel D: percent of Gb3(%) remaining in the kidney relative to the GLA KO male mice treated with DPBS. Error bars indicate standard deviation.

FIG. 5 shows the GLA activity and IHC staining for GLA and Gb3 in the liver of GLA KO mice administered with the rAAVs. Panel A: GLA activity in liver; Panel B: IHC staining with anti-human GLA antibody in liver; and Panel C: IHC staining with anti-mouse Gb3 antibody in liver.

FIG. 6 shows the GLA activity and IHC staining for GLA in the heart of GLA KO mice administered with the rAAVs. Panel A: GLA activity in heart; and Panel B: IHC staining with anti-human GLA antibody in heart.

FIG. 7 shows the GLA activity and IHC staining for GLA in the brain and spinal cord of GLA KO mice administered with the rAAVs. Panel A: GLA activities in brain; Panel B: GLA activities in spinal cord; and Panel C: IHC staining with anti-human GLA antibody in brain and spinal cord.

FIG. 8 shows the GLA activity in liver (A), heart (B), kidney (C) and plasma (D) of GLA KO mice administered with rAAVs 108, 109 and 110.

FIG. 9 shows the copies of vector genome (A), mRNA level of GLA (B) and GLA activity (C) in liver, heart and kidney of GLA KO mice administered with rAAVs 108, 109 and 110.

FIG. 10 shows the Gb3 concentrations in liver (A), heart (B), kidney (C) and plasma (D) of GLA KO mice administered with rAAVs 108, 109 and 110 (n=2). The Gb3 concentration in the GLA KO mice administered with DPBS (negative control) is indicated as 100%, and the other groups are indicated relative to the negative control.

FIG. 11 shows the latency periods of the mice.

FIG. 12 shows the body weight gain of the mice treated with DPBS, rAAV 106 or 108. A: female, B: male.

FIG. 13 shows the vector genome copies (A) and mRNA levels of GLA (B) in liver, heart, kidney, brain and spleen.

FIG. 14 shows the GLA activities in liver (A), heart (B), kidney (C) and spleen (D) of the mice. The GLA activities in male WT mice treated with DPBS are normalized as 1×, and the GLA activities of other groups are expressed relatively.

FIG. 15 shows the GLA activities in plasma of the mice over a 24-week time period, A: female, B: male.

FIG. 16 shows the Gb3 concentrations and GLA activities (n=2) in liver (A and B) and heart (C and D).

FIG. 17 shows the Gb3 concentrations and GLA activities (n=2) in kidney (A and B) and plasma (C and D).

FIG. 18 shows the results of the ALT (A) and AST (B) analysis.

FIG. 19 shows the effect of rAAV 108 on the body weight gain (A), GLA activities in plasma (B), BUN levels (C) and clasping status (D-F) in Fabry Aggravated models.

FIG. 20 shows the effect of rAAV 108 on the urine albumin (A), urine creatinine (B), the ratio of albumin to creatinine in urine (C) and urine osmolality (D) in Fabry Aggravated models.

### DETAILED DESCRIPTION

### 1. Definitions

Unless otherwise indicated, all terms used herein have the same meaning as they would to one skilled in the art, and the practice of the present invention will employ conventional techniques of microbiology and recombinant DNA technology, which are within the knowledge of those of skill in the art.

As used herein, “GLA” and “α-Gal A” are exchangeable when referring to a protein, and mean alpha galactosidase A. For example, the “GLA activity” in a tissue means the activity of alpha galactosidase A therein. “GLA” may also mean the gene encoding alpha galactosidase A when referring to a nucleic acid.

Adeno-associated virus (AAV) is a member of Parvoviridae family. It is a simple single-stranded DNA virus, and requires a helper virus (such as adenovirus) for replication. The genome of a wildtype AAV contains approximately 4.7 kilobases (kb), comprising the cap and rep genes between two inverted terminal repeat (ITR) sequences, approximately 145 nucleotides in length, with interrupted palindromic sequences that can fold into hairpin structures that function as primers during initiation of DNA replication. The cap gene encodes the viral capsid protein, and the rep gene is involved in the replication and integration of AAV.

AAV can infect a variety of cells, and the viral DNA can be integrated into human chromosome 19 in the presence of the rep product.

As used herein, the term “inverted terminal repeats” or “ITRs” as used herein refers to AAV viral cis-elements named due to their symmetry. These elements are essential for efficient multiplication of an AAV genome. In the present invention, the term “ITR” refers to ITRs of known natural AAV serotypes, to chimeric ITRs formed by the fusion of ITR elements derived from different serotypes, and to functional variant thereof.

The production of a recombinant AAV particle may involve three plasmids, a transgene plasmid comprising an expression construct for expressing an exogenous polynucleotide, a packaging plasmid encoding the REP and/or CAP proteins, and a helper plasmid.

As used herein, the term “expression construct” refers to a single-stranded or double-stranded polynucleotide, which is isolated from a naturally occurring gene or modified to contain a nucleic acid segment that does not naturally occur. The expression construct may contain the control sequences required to express the coding sequence of the present invention.

As used herein, the term “polynucleotide” usually refers to generally a nucleic acid molecule (e.g., 100 bases and up to 30 kilobases in length) and a sequence that is either complementary (antisense) or identical (sense) to the sequence of a messenger RNA (mRNA) or miRNA fragment or molecule. The term can also refer to DNA or RNA molecules that are either transcribed or non-transcribed.

The term “exogenous polynucleotide” as used herein refers to a nucleotide sequence that does not originate from the host in which it is placed. It may be identical to the host's DNA or heterologous. An example is a sequence of interest inserted into a vector. Such exogenous DNA sequences may be derived from a variety of sources including DNA, cDNA, synthetic DNA, and RNA. Exogenous polynucleotides also encompass DNA sequences that encode antisense oligonucleotides.

As used herein, the term “expression” includes any step involved in the production of a polypeptide, including but not limited to transcription, post-transcriptional modification, translation, post-translational modification, and secretion.

A “control sequence” includes all elements necessary or beneficial for the expression of the polynucleotide encoding the polypeptide of the present invention. Each control sequence may be natural or foreign to the nucleotide sequence encoding the polypeptide, or natural or foreign to each other. Such control sequences include, but are not limited to, leader sequence, polyadenylation sequence, propeptide sequence, promoter, enhancer, signal peptide sequence, and transcription terminator. At a minimum, control sequences include a promoter and signals for the termination of transcription and translation.

For example, the control sequence may be a suitable promoter sequence, a nucleotide sequence recognized by the host cell to express the polynucleotide encoding the polypeptide of the present invention. The promoter sequence contains a transcription control sequence that mediates the expression of the polypeptide. The promoter may be any nucleotide sequence that exhibits transcriptional activity in the selected host cell, for example, lac operon of E. coli.

The promoters also include mutant, modified and hybrid promoters, and can be obtained from genes encoding extracellular or intracellular polypeptides, which are homologous or heterologous to the host cell.

In some cases, an intron can be included in the construct to improve the expression of the coding sequence. A “modified” intron comprises a modification such as substitution, insertion or deletion of one or more nucleotides in an internal region of an initial intron.

As used herein, the term “operably linked” herein refers to a configuration in which a control sequence is placed at an appropriate position relative to the coding sequence of the polynucleotide sequence, whereby the control sequence directs the expression of the polypeptide coding sequence.

The polynucleotide encoding the GLA protein can be subjected to various manipulations to improve the expression of the polypeptide. Before the insertion thereof into a vector, manipulation of the polynucleotide according to the expression vector or the host, such as codon optimization, is desirable or necessary.

The term “recombinant” as used herein refers to nucleic acids, vectors, polypeptides, or proteins that have been generated using DNA recombination (cloning) methods and are distinguishable from native or wild-type nucleic acids, vectors, polypeptides, or proteins.

The terms “polypeptide” and “protein” are used interchangeably herein and refer to a polymer of amino acids and includes full-length proteins and fragments thereof.

As used herein, the term “host cell” refers to, for example microorganisms, yeast cells, insect cells, and mammalian cells, that can be, or have been, used as recipients of rAAV vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation.

The term “pharmaceutically acceptable” as used herein refers to molecular entities and compositions that are physiologically tolerable and do not typically produce toxicity or an allergic or similar untoward reaction, such as gastric upset, dizziness and the like, when administered to a human.

The term “subject” as used herein includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

### 2. The Polynucleotide or Construct for Expressing GLA

Gene therapy aims to correct defective genes that underlie the development of diseases, and to introduce exogenous gene into the cell of interest in a subject to express the product of the exogenous gene that is useful for treating a certain disease, such as Fabry disease. A common approach for this purpose involves the delivery of a functional gene such as GLA to the nucleus. This gene may then be inserted into the genome of the cell of interest or may remain episomal. Delivery of a functional gene to a subject's target cells can be carried out via numerous methods, including the use of viral vectors. Among the many viral vectors available (e.g, retrovirus, lentivirus, adenovirus, and the like), AAV is gaining popularity as a versatile vector in gene therapy.

Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, e.g., interferon-mediated responses; (iii) wild-type viruses have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected). AAV vectors can also be produced at high titer and it has been reported that intra-arterial, intra-venous, or intra-peritoneal injections allow gene transfer to significant muscle regions in rodents through a single injection.

The present invention thus intends to provide a polynucleotide of interest, an expression construct, or a vector comprising the polynucleotide of interest or the expression construct, for expressing GLA in a subject. In some embodiments, the GLA can be from any animal species, including but not limited to, human, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like.

In some embodiments, the GLA is human GLA. In some embodiments, the GLA comprise the amino acid sequence of SEQ ID NO: 12 or an allelic variant thereof. In some embodiments, the GLA is a functional GLA polypeptide, such as a GLA polypeptide having at least 10%, 20%, 30%, 40%, 50%, 60% 70%, 80%, 90%, 100% or more activity of the polypeptide of SEQ ID NO: 12.

In some embodiments, the polynucleotide encoding GLA is codon-optimized to improve the expression.

In some embodiments, the polynucleotide comprises a nucleotide sequence selected from SEQ ID NOs: 1, 2, 3 and 4. Surprisingly, the inventors found that the polynucleotide with a nucleotide sequence of SEQ ID NO: 4 achieves a higher expression of GLA than SEQ ID NOs: 1, 2 and 3 in various tissues, including liver, heart, kidney, brain, spinal cord, plasma, etc.

In some embodiments, the polynucleotide comprises SEQ ID NO: 1 or a nucleotide sequence at least 80%, 85%, 90%, 95%, 96%, 97%, 98%, or 99% identical to SEQ ID NO: 1. In some embodiments, the polynucleotide comprises a nucleotide sequence at least 80%, 85%, 90%, 95%, 96%, 97%, 98%, or 99% identical to SEQ ID NO: 1, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the polynucleotide comprises a nucleotide sequence at least 80%, 85%, 90%, 95%, 96%, 97%, 98%, or 99% identical to SEQ ID NO: 1, and encodes the polypeptide of SEQ ID NO: 12 or an allelic variant thereof.

In some embodiments, the polynucleotide comprises SEQ ID NO: 2 or a nucleotide sequence at least 80%, 85%, 90%, 95%, 96%, 97%, 98%, or 99% identical to SEQ ID NO: 2. In some embodiments, the polynucleotide comprises a nucleotide sequence at least 80%, 85%, 90%, 95%, 96%, 97%, 98%, or 99% identical to SEQ ID NO: 2, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the polynucleotide comprises a nucleotide sequence at least 80%, 85%, 90%, 95%, 96%, 97%, 98%, or 99% identical to SEQ ID NO: 2, and encodes the polypeptide of SEQ ID NO: 12 or an allelic variant thereof.

In some embodiments, the polynucleotide comprises SEQ ID NO: 3 or a nucleotide sequence at least 80%, 85%, 90%, 95%, 96%, 97%, 98%, or 99% identical to SEQ ID NO: 3. In some embodiments, the polynucleotide comprises a nucleotide sequence at least 80%, 85%, 90%, 95%, 96%, 97%, 98%, or 99% identical to SEQ ID NO: 3, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the polynucleotide comprises a nucleotide sequence at least 80%, 85%, 90%, 95%, 96%, 97%, 98%, or 99% identical to SEQ ID NO: 3, and encodes the polypeptide of SEQ ID NO: 12 or an allelic variant thereof.

In some embodiments, the polynucleotide comprises SEQ ID NO: 4 or a nucleotide sequence at least 80%, 85%, 90%, 95%, 96%, 97%, 98%, or 99% identical to SEQ ID NO: 4. In some embodiments, the polynucleotide comprises a nucleotide sequence at least 80%, 85%, 90%, 95%, 96%, 97%, 98%, or 99% identical to SEQ ID NO: 4, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the polynucleotide comprises a nucleotide sequence at least 80%, 85%, 90%, 95%, 96%, 97%, 98%, or 99% identical to SEQ ID NO: 4 and encodes the polypeptide of SEQ ID NO: 12 or an allelic variant thereof.

The present invention also provides an expression construct for expressing GLA, which comprises a polynucleotide of interest encoding GLA operably linked to a promoter. In some embodiments, the GLA is human GLA. In some embodiments, the GLA comprises the amino acid sequence of SEQ ID NO: 12 or an allelic variant thereof.

In some embodiments, the nucleotide sequence is codon-optimized to improve the expression. In some embodiments, the codon-optimized nucleotide sequence is selected from SEQ ID NOs: 1, 2, 3 and 4. Surprisingly, the inventors found that the polynucleotide with a nucleotide sequence of SEQ ID NO: 4 achieves a higher expression of GLA than SEQ ID NOs: 1, 2 and 3 in various tissues, including liver, heart, kidney, brain, spinal cord, plasma, etc.

In some embodiments, the nucleotide sequence is SEQ ID NO: 1 or at least 80%, 85%, 90%, 95%, 96%, 97%, 98%, or 99% identical to SEQ ID NO: 1. In some embodiments, the nucleotide sequence is at least 80%, 85%, 90%, 95%, 96%, 97%, 98%, or 99% identical to SEQ ID NO: 1, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 1, and encodes the polypeptide of SEQ ID NO: 12 or an allelic variant thereof.

In some embodiments, the nucleotide sequence is SEQ ID NO: 2 or at least 80%, 85%, 90%, 95%, 96%, 97%, 98%, or 99% identical to SEQ ID NO: 2. In some embodiments, the nucleotide sequence is at least 80%, 85%, 90%, 95%, 96%, 97%, 98%, or 99% identical to SEQ ID NO: 2, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 2, and encodes the polypeptide of SEQ ID NO: 12 or an allelic variant thereof.

In some embodiments, the nucleotide sequence is SEQ ID NO: 3 or at least 80%, 85%, 90%, 95%, 96%, 97%, 98%, or 99% identical to SEQ ID NO: 3. In some embodiments, the nucleotide sequence is at least 80%, 85%, 90%, 95%, 96%, 97%, 98%, or 99% identical to SEQ ID NO: 3, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 3, and encodes the polypeptide of SEQ ID NO: 12 or an allelic variant thereof.

In some embodiments, the nucleotide sequence is SEQ ID NO: 4 or at least 80%, 85%, 90%, 95%, 96%, 97%, 98%, or 99% identical to SEQ ID NO: 4. In some embodiments, the nucleotide sequence is at least 80%, 85%, 90%, 95%, 96%, 97%, 98%, or 99% identical to SEQ ID NO: 4, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 4 and encodes the polypeptide of SEQ ID NO: 12 or an allelic variant thereof.

The construct will generally be transferred to mammalian cells (such as human cells) for expression. Such constructs often include promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene.

In some embodiments, the promoter is a constitutive promoter. In some embodiments, the promoter is a chicken beta-actin core promoter. In some embodiments, the promoter comprises SEQ ID NO: 10 or a nucleotide sequence at least 70%, 75%, 80%, 85%, 90%, 95%, 96%, 97%, 98%, or 99% identical to SEQ ID NO: 10.

In some embodiments, the expression construct further comprises an enhancer. In some embodiments, the enhancer is upstream of the promoter. In some embodiments, the enhancer is a cytomegalovirus (CMV) early enhancer. In some embodiments, the enhancer comprises SEQ ID NO: 9, or a nucleotide sequence at least 70%, 75%, 80%, 85%, 90%, 95%, 96%, 97% 98%, or 99% identical to SEQ ID NO: 9.

It is also desirable to include an intron (e.g., an intron derived from a eukaryotic organism or an artificial intron) in the construct to increase the expression in eukaryotic cells. In some embodiments, the expression construct further comprises an intron. In some embodiments, the intron is upstream of the nucleotide encoding GLA. In some embodiments, the intron is downstream of the promoter.

The inventor surprisingly found that the incorporation of a longer intron led to an increased expression. In some embodiments, the intron is at least 200 nucleotides in length. In some embodiments, the intron is up to about 250, 300, 350, 400, 450, 500, 550, 600, 650, 700, 750, 800, 850, 900, 950, 1,000, 1,050, 1,100, 1,200, 1,300, 1,400, or 1,500 nucleotides in length. In some embodiments, the intron is 200-1,500, 200-1,400, 250-1,300, 300-1,200, 400-1,300, 400-1,200, or 400-1,100 nucleotides in length.

In some embodiments, the intron is a chicken beta-actin (CBA) chimeric intron, which is, e.g., set forth in SEQ ID NO: 5. In some embodiments, the intron is a modified CBA chimeric intron. In some embodiments, the modified intron comprises a modification such as substitution, insertion or deletion of one or more nucleotides in a region of 662 nucleotides between positions 223 and 893 as compared to SEQ ID NO: 5. In some embodiments, the modification is a deletion. In some embodiments, the modification is a deletion of 662 consecutive nucleotides, e.g. nucleotides 224-885, 225-886, 226-887, 227-888, 228-889, 229-890, 230-891, or 231-892 In some embodiments, the modified CBA chimeric intron is set forth in SEQ ID NO: 6.

In some embodiments, the modified CBA chimeric intron, as compared to SEQ ID NO: 5, comprises a deletion of a fragment, which is up to 662 bp in length, from the region between positions 223 and 893 of SEQ ID NO: 5. In some embodiments, the fragment is 2-660, 5-650, 10-600, 25-500, 50-400, 75-300, 100-200 nucleotides in length. In some embodiments, the fragment is at least 2, 3, 4, 5, 6, 7, 8, 9, 10, 15, 20, 25, 30, 35, 40, 45, 50, 75, 100, 125, 150, 175, 200, 250, 300, 350, 400, 450, 500, 550, 600, 650 nucleotides and up to 662 nucleotides in length. In some embodiments, the fragment comprises 2-660, 5-650, 10-600, 25-500, 50-400, 75-300, 100-200 consecutive nucleotides between positions 223 and 893 of SEQ ID NO: 5. In some embodiments, the fragment comprises at least 2, 3, 4, 5, 6, 7, 8, 9, 10, 15, 20, 25, 30, 35, 40, 45, 50, 75, 100, 125, 150, 175, 200, 250, 300, 350, 400, 450, 500, 550, 600, 650, and up to 662 consecutive nucleotides between positions 223 and 893 of SEQ ID NO: 5.

In some embodiments, the intron comprises SEQ ID NO: 5 or 6, or a nucleotide sequence at least 70%, 75%, 80%, 85%, 90%, 95%, 96%, 97%, 98%, or 99% identical to SEQ ID NO: 5 or 6.

The expression construct further comprises a polyadenylation signal sequence for the processing of the transcript. In some embodiments, the construct comprises a polyadenylation signal sequence downstream of the nucleotide sequence encoding GLA. In some embodiments, the polyadenylation signal sequence comprises SEQ ID NO: 11, or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′, an enhancer, a promoter, an intron, a polynucleotide of interest encoding GLA, and a polyadenylation signal sequence.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 1 or a nucleotide sequence at least 80%, 85%, 90%, 95%, 96%, 97%, 98%, or 99% identical to SEQ ID NO: 1, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 1 or a nucleotide sequence at least 80%, 85%, 90%, 95%, 96%, 97%, 98%, or 99% identical to SEQ ID NO: 1, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 2 or a nucleotide sequence at least 80%, 85%, 90%, 95%, 96%, 97%, 98%, or 99% identical to SEQ ID NO: 2, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 2 or a nucleotide sequence at least 80%, 85%, 90%, 95%, 96%, 97%, 98%, or 99% identical to SEQ ID NO: 2, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 3 or a nucleotide sequence at least 80%, 85%, 90%, 95%, 96%, 97%, 98%, or 99% identical to SEQ ID NO: 3, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 3 or a nucleotide sequence at least 80%, 85%, 90%, 95%, 96%, 97%, 98%, or 99% identical to SEQ ID NO: 3, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 4 or a nucleotide sequence at least 80%, 85%, 90%, 95%, 96%, 97%, 98%, or 99% identical to SEQ ID NO: 4, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 4 or a nucleotide sequence at least 80%, 85%, 90%, 95%, 96%, 97%, 98%, or 99% identical to SEQ ID NO: 4, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 1 or a nucleotide sequence at least 80%, 85%, 90%, 95%, 96%, 97%, 98%, or 99% identical to SEQ ID NO: 1, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 1 or a nucleotide sequence at least 80%, 85%, 90%, 95%, 96%, 97%, 98%, or 99% identical to SEQ ID NO: 1, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 2 or a nucleotide sequence at least 80%, 85%, 90%, 95%, 96%, 97%, 98%, or 99% identical to SEQ ID NO: 2, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 2 or a nucleotide sequence at least 80%, 85%, 90%, 95%, 96%, 97%, 98%, or 99% identical to SEQ ID NO: 2, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 3 or a nucleotide sequence at least 80%, 85%, 90%, 95%, 96%, 97%, 98%, or 99% identical to SEQ ID NO: 3, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 3 or a nucleotide sequence at least 80%, 85%, 90%, 95%, 96%, 97%, 98%, or 99% identical to SEQ ID NO: 3, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 4 or a nucleotide sequence at least 80%, 85%, 90%, 95%, 96%, 97%, 98%, or 99% identical to SEQ ID NO: 4, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 4 or a nucleotide sequence at least 80%, 85%, 90%, 95%, 96%, 97%, 98%, or 99% identical to SEQ ID NO: 4, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′, SEQ ID NO: 9, SEQ ID NO: 10, SEQ ID NO: 5, SEQ ID NO: 1 or a nucleotide sequence at least 80%, 85%, 90%, 95%, 96%, 97%, 98%, or 99% identical to SEQ ID NO: 1, and SEQ ID NO: 11. In some embodiments, the expression construct comprises, in the order of 5′ to 3′, SEQ ID NO: 9, SEQ ID NO: 10, SEQ ID NO: 6, SEQ ID NO: 1 or a nucleotide sequence at least 80%, 85%, 90%, 95%, 96%, 97%, 98%, or 99% identical to SEQ ID NO: 1, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 2 or a nucleotide sequence at least 80%, 85%, 90%, 95%, 96%, 97%, 98%, or 99% identical to SEQ ID NO: 2, and SEQ ID NO: 11. In some embodiments, the expression construct comprises, in the order of 5′ to 3′, SEQ ID NO: 9, SEQ ID NO: 10, SEQ ID NO: 6, SEQ ID NO: 2 or a nucleotide sequence at least 80%, 85%, 90%, 95%, 96%, 97%, 98%, or 99% identical to SEQ ID NO: 2, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 3 or a nucleotide sequence at least 80%, 85%, 90%, 95%, 96%, 97%, 98%, or 99% identical to SEQ ID NO: 3, and SEQ ID NO: 11. In some embodiments, the expression construct comprises, in the order of 5′ to 3′, SEQ ID NO: 9, SEQ ID NO: 10, SEQ ID NO: 6, SEQ ID NO: 3 or a nucleotide sequence at least 80%, 85%, 90%, 95%, 96%, 97%, 98%, or 99% identical to SEQ ID NO: 3, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 4 or a nucleotide sequence at least 80%, 85%, 90%, 95%, 96%, 97%, 98%, or 99% identical to SEQ ID NO: 4, and SEQ ID NO: 11. In some embodiments, the expression construct comprises, in the order of 5′ to 3′, SEQ ID NO: 9, SEQ ID NO: 10, SEQ ID NO: 6, SEQ ID NO: 4 or a nucleotide sequence at least 80%, 85%, 90%, 95%, 96%, 97%, 98%, or 99% identical to SEQ ID NO: 4, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 1 or a nucleotide sequence at least 80%, 85%, 90%, 95%, 96%, 97%, 98%, or 99% identical to SEQ ID NO: 1, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 1 or a nucleotide sequence at least 80%, 85%, 90%, 95%, 96%, 97%, 98%, or 99% identical to SEQ ID NO: 1, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 2 or a nucleotide sequence at least 80%, 85%, 90%, 95%, 96%, 97%, 98%, or 99% identical to SEQ ID NO: 2, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 2 or a nucleotide sequence at least 80%, 85%, 90%, 95%, 96%, 97%, 98%, or 99% identical to SEQ ID NO: 2, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 3 or a nucleotide sequence at least 80%, 85%, 90%, 95%, 96%, 97%, 98%, or 99% identical to SEQ ID NO: 3, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 3 or a nucleotide sequence at least 80%, 85%, 90%, 95%, 96%, 97%, 98%, or 99% identical to SEQ ID NO: 3, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 4 or a nucleotide sequence at least 80%, 85%, 90%, 95%, 96%, 97%, 98%, or 99% identical to SEQ ID NO: 4, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 4 or a nucleotide sequence at least 80%, 85%, 90%, 95%, 96%, 97%, 98%, or 99% identical to SEQ ID NO: 4, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 1, and SEQ ID NO: 11. In some embodiments, the expression construct comprises, in the order of 5′ to 3′, SEQ ID NO: 9, SEQ ID NO: 10, SEQ ID NO: 6, SEQ ID NO: 1, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 2, and SEQ ID NO: 11. In some embodiments, the expression construct comprises, in the order of 5′ to 3′, SEQ ID NO: 9, SEQ ID NO: 10, SEQ ID NO: 6, SEQ ID NO: 2, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 3, and SEQ ID NO: 11. In some embodiments, the expression construct comprises, in the order of 5′ to 3′, SEQ ID NO: 9, SEQ ID NO: 10, SEQ ID NO: 6, SEQ ID NO: 3, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 4, and SEQ ID NO: 11. In some embodiments, the expression construct comprises, in the order of 5′ to 3′, SEQ ID NO: 9, SEQ ID NO: 10, SEQ ID NO: 6, SEQ ID NO: 4, and SEQ ID NO: 11.

### 3. Recombinant AAV, Pharmaceutical Composition and Host Cell

The present invention further provides a recombinant AAV (rAAV) comprising a genome comprising the expression construct of the invention. In some embodiments, the expression construct flanked by 5′ and 3′ inverted terminal repeats (ITRs) of AAV.

In some embodiments, the rAAV of the invention can be selected from human serotype 1 AAV (hAAV1), hAAV2, hAAV3, hAAV4, hAAV5, hAAV6, hAAV7, hAAV8, hAAV9, hAAV10, and hAAV11. In some embodiments, the rAAV is hAAV9.

In some embodiment, the genome of the rAAV comprises an expression construct flanked by 5′ and 3′ ITRs of AAV.

In some embodiments, the construct comprises a nucleotide sequence encoding GLA that is operably linked to a promoter. In some embodiments, the GLA is human GLA. In some embodiments, the GLA comprise the amino acid sequence of SEQ ID NO: 12 or an allelic variant thereof.

In some embodiments, the nucleotide sequence is codon-optimized to improve the expression. In some embodiments, the codon-optimized nucleotide sequence is selected from SEQ ID NOs: 1, 2, 3 and 4. Surprisingly, the inventors found that the polynucleotide with a nucleotide sequence of SEQ ID NO: 4 achieves a higher expression of GLA than SEQ ID NOs: 1, 2 and 3 in various tissues, including liver, heart, kidney, brain, spinal cord, plasma, etc.

In some embodiments, the nucleotide sequence is SEQ ID NO: 1 or at least 80%, 85%, 90%, 95%, 96%, 97%, 98%, or 99% identical to SEQ ID NO: 1. In some embodiments, the nucleotide sequence is at least 80%, 85%, 90%, 95%, 96%, 97%, 98%, or 99% identical to SEQ ID NO: 1, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 1, and encodes the polypeptide of SEQ ID NO: 12 or an allelic variant thereof.

In some embodiments, the nucleotide sequence is SEQ ID NO: 2 or at least 80%, 85%, 90%, 95%, 96%, 97%, 98%, or 99% identical to SEQ ID NO: 2. In some embodiments, the nucleotide sequence is at least 80%, 85%, 90%, 95%, 96%, 97%, 98%, or 99% identical to SEQ ID NO: 2, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 2, and encodes the polypeptide of SEQ ID NO: 12 or an allelic variant thereof.

In some embodiments, the nucleotide sequence is SEQ ID NO: 3 or at least 80%, 85%, 90%, 95%, 96%, 97%, 98%, or 99% identical to SEQ ID NO: 3. In some embodiments, the nucleotide sequence is at least 80%, 85%, 90%, 95%, 96%, 97%, 98%, or 99% identical to SEQ ID NO: 3, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 3, and encodes the polypeptide of SEQ ID NO: 12 or an allelic variant thereof.

In some embodiments, the nucleotide sequence is SEQ ID NO: 4 or at least 80%, 85%, 90%, 95%, 96%, 97%, 98%, or 99% identical to SEQ ID NO: 4. In some embodiments, the nucleotide sequence is at least 80%, 85%, 90%, 95%, 96%, 97%, 98%, or 99% identical to SEQ ID NO: 4, and encodes a functional GLA polypeptide, such as a GLA polypeptide having at least 10%, 20%, 30%, 40%, 50%, 60% 70%, 80%, 90%, 100% or more activity of the polypeptide of SEQ ID NO: 12. In some embodiments, the nucleotide sequence is at least 80%, 85%, 90%, 95%, 96%, 97%, 98%, or 99% identical to SEQ ID NO: 4 and encodes the polypeptide of SEQ ID NO: 12 or an allelic variant thereof.

In some embodiments, the promoter is a constitutive promoter. In some embodiments, the promoter is a chicken beta-actin core promoter. In some embodiments, the promoter comprises SEQ ID NO: 10 or a nucleotide sequence at least 70%, 75%, 80%, 85%, 90%, 95%, 96%, 97%, 98%, or 99% identical to SEQ ID NO: 10.

In some embodiments, the expression construct further comprises an enhancer. In some embodiments, the enhancer is upstream of the promoter. In some embodiments, the enhancer is a cytomegalovirus (CMV) early enhancer. In some embodiments, the enhancer comprises SEQ ID NO: 9, or a nucleotide sequence at least 70%, 75%, 80%, 85%, 90%, 95%, 96%, 97% 98%, or 99% identical to SEQ ID NO: 9.

It is also desirable to include an intron (e.g., an intron derived from a eukaryotic organism or an artificial intron) in the construct to increase the expression in eukaryotic cells. In some embodiments, the expression construct further comprises an intron. In some embodiments, the intron is upstream of the nucleotide encoding GLA. In some embodiments, the intron is downstream of the promoter.

The inventor surprisingly found that the incorporation of a longer intron led to an increased expression. In some embodiments, the intron is at least 200 nucleotides in length. In some embodiments, the intron is up to about 250, 300, 350, 400, 450, 500, 550, 600, 650, 700, 750, 800, 850, 900, 950, 1,000, 1,050, 1,100, 1,200, 1,300, 1,400, or 1,500 nucleotides in length. In some embodiments, the intron is 200-1,500, 200-1,400, 250-1,300, 300-1,200, 400-1,300, 400-1,200, or 400-1,100 nucleotides in length.

In some embodiments, the intron is a chicken beta-actin (CBA) chimeric intron, which is, e.g., set forth in SEQ ID NO: 5. In some embodiments, the intron is a modified CBA chimeric intron. In some embodiments, the modified intron comprises a modification such as substitution, insertion or deletion of one or more nucleotides in a region of 662 nucleotides between positions 223 and 893 as compared to SEQ ID NO: 5. In some embodiments, the modification is a deletion. In some embodiments, the modification is a deletion of 662 consecutive nucleotides, e.g. nucleotides 224-885, 225-886, 226-887, 227-888, 228-889, 229-890, 230-891, or 231-892 In some embodiments, the modified CBA chimeric intron is set forth in SEQ ID NO: 6.

In some embodiments, the modified CBA chimeric intron, as compared to SEQ ID NO: 5, comprises a deletion of a fragment, which is up to 662 bp in length, from the region between positions 223 and 893 of SEQ ID NO: 5. In some embodiments, the fragment is 2-660, 5-650, 10-600, 25-500, 50-400, 75-300, 100-200 nucleotides in length. In some embodiments, the fragment is at least 2, 3, 4, 5, 6, 7, 8, 9, 10, 15, 20, 25, 30, 35, 40, 45, 50, 75, 100, 125, 150, 175, 200, 250, 300, 350, 400, 450, 500, 550, 600, 650 nucleotides and up to 662 nucleotides in length. In some embodiments, the fragment comprises 2-660, 5-650, 10-600, 25-500, 50-400, 75-300, 100-200 consecutive nucleotides between positions 223 and 893 of SEQ ID NO: 5. In some embodiments, the fragment comprises at least 2, 3, 4, 5, 6, 7, 8, 9, 10, 15, 20, 25, 30, 35, 40, 45, 50, 75, 100, 125, 150, 175, 200, 250, 300, 350, 400, 450, 500, 550, 600, 650, and up to 662 consecutive nucleotides between positions 223 and 893 of SEQ ID NO: 5.

In some embodiments, the intron comprises SEQ ID NO: 5 or 6, or a nucleotide sequence at least 70%, 75%, 80%, 85%, 90%, 95%, 96%, 97%, 98%, or 99% identical to SEQ ID NO: 5 or 6.

The expression construct further comprises a polyadenylation signal sequence for the processing of the transcript. In some embodiments, the construct comprises a polyadenylation signal sequence downstream of the nucleotide sequence encoding GLA. In some embodiments, the polyadenylation signal sequence comprises SEQ ID NO: 11, or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′, an enhancer, a promoter, an intron, a nucleotide encoding GLA, and a polyadenylation signal sequence.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 1 or a nucleotide sequence at least 80%, 85%, 90%, 95%, 96%, 97%, 98%, or 99% identical to SEQ ID NO: 1, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 1 or a nucleotide sequence at least 80%, 85%, 90%, 95%, 96%, 97%, 98%, or 99% identical to SEQ ID NO: 1, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 2 or a nucleotide sequence at least 80%, 85%, 90%, 95%, 96%, 97%, 98%, or 99% identical to SEQ ID NO: 2, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 2 or a nucleotide sequence at least 80%, 85%, 90%, 95%, 96%, 97%, 98%, or 99% identical to SEQ ID NO: 2, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 3 or a nucleotide sequence at least 80%, 85%, 90%, 95%, 96%, 97%, 98%, or 99% identical to SEQ ID NO: 3, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 3 or a nucleotide sequence at least 80%, 85%, 90%, 95%, 96%, 97%, 98%, or 99% identical to SEQ ID NO: 3, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 4 or a nucleotide sequence at least 80%, 85%, 90%, 95%, 96%, 97%, 98%, or 99% identical to SEQ ID NO: 4, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 4 or a nucleotide sequence at least 80%, 85%, 90%, 95%, 96%, 97%, 98%, or 99% identical to SEQ ID NO: 4, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 1 or a nucleotide sequence at least 80%, 85%, 90%, 95%, 96%, 97%, 98%, or 99% identical to SEQ ID NO: 1, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 1 or a nucleotide sequence at least 80%, 85%, 90%, 95%, 96%, 97%, 98%, or 99% identical to SEQ ID NO: 1, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 2 or a nucleotide sequence at least 80%, 85%, 90%, 95%, 96%, 97%, 98%, or 99% identical to SEQ ID NO: 2, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 2 or a nucleotide sequence at least 80%, 85%, 90%, 95%, 96%, 97%, 98%, or 99% identical to SEQ ID NO: 2, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 3 or a nucleotide sequence at least 80%, 85%, 90%, 95%, 96%, 97%, 98%, or 99% identical to SEQ ID NO: 3, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 3 or a nucleotide sequence at least 80%, 85%, 90%, 95%, 96%, 97%, 98%, or 99% identical to SEQ ID NO: 3, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 5 or a nucleotide sequence at least 70%, 75%, 80%, 85%, 90%, 95%, 96%, 97%, 98%, or 99% identical to SEQ ID NO: 5,
SEQ ID NO: 4 or a nucleotide sequence at least 80%, 85%, 90%, 95%, 96%, 97%, 98%, or 99% identical to SEQ ID NO: 4, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′,
SEQ ID NO: 9 or a nucleotide sequence at least 70%, 75%, 80%, 85%, 90%, 95%, 96%, 97%, 98%, or 99% identical to SEQ ID NO: 9,
SEQ ID NO: 10 or a nucleotide sequence at least 70%, 75%, 80%, 85%, 90%, 95%, 96%, 97%, 98%, or 99% identical to SEQ ID NO: 10,
SEQ ID NO: 6 or a nucleotide sequence at least 70%, 75%, 80%, 85%, 90%, 95%, 96%, 97%, 98%, or 99% identical to SEQ ID NO: 6,
SEQ ID NO: 4 or a nucleotide sequence at least 80%, 85%, 90%, 95%, 96%, 97%, 98%, or 99% identical to SEQ ID NO: 4, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12, and
SEQ ID NO: 11 or a nucleotide sequence at least 70%, 75%, 80%, 85%, 90%, 95%, 96%, 97%, 98%, or 99% identical to SEQ ID NO: 11.

In some embodiments, the expression construct comprises, in the order of 5′ to 3′, SEQ ID NO: 9, SEQ ID NO: 10, SEQ ID NO: 5, SEQ ID NO: 1 or a nucleotide sequence at least 80%, 85%, 90%, 95%, 96%, 97%, 98%, or 99% identical to SEQ ID NO: 1, and SEQ ID NO: 11. In some embodiments, the expression construct comprises, in the order of 5′ to 3′, SEQ ID NO: 9, SEQ ID NO: 10, SEQ ID NO: 6, SEQ ID NO: 1 or a nucleotide sequence at least 80%, 85%, 90%, 95%, 96%, 97%, 98%, or 99% identical to SEQ ID NO: 1, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 2 or a nucleotide sequence at least 80%, 85%, 90%, 95%, 96%, 97%, 98%, or 99% identical to SEQ ID NO: 2, and SEQ ID NO: 11. In some embodiments, the expression construct comprises, in the order of 5′ to 3′, SEQ ID NO: 9, SEQ ID NO: 10, SEQ ID NO: 6, SEQ ID NO: 2 or a nucleotide sequence at least 80%, 85%, 90%, 95%, 96%, 97%, 98%, or 99% identical to SEQ ID NO: 2, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 3 or a nucleotide sequence at least 80%, 85%, 90%, 95%, 96%, 97%, 98%, or 99% identical to SEQ ID NO: 3, and SEQ ID NO: 11. In some embodiments, the expression construct comprises, in the order of 5′ to 3′, SEQ ID NO: 9, SEQ ID NO: 10, SEQ ID NO: 6, SEQ ID NO: 3 or a nucleotide sequence at least 80%, 85%, 90%, 95%, 96%, 97%, 98%, or 99% identical to SEQ ID NO: 3, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 4 or a nucleotide sequence at least 80%, 85%, 90%, 95%, 96%, 97%, 98%, or 99% identical to SEQ ID NO: 4, and SEQ ID NO: 11. In some embodiments, the expression construct comprises, in the order of 5′ to 3′, SEQ ID NO: 9, SEQ ID NO: 10, SEQ ID NO: 6, SEQ ID NO: 4 or a nucleotide sequence at least 80%, 85%, 90%, 95%, 96%, 97%, 98%, or 99% identical to SEQ ID NO: 4, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 1 or a nucleotide sequence at least 80%, 85%, 90%, 95%, 96%, 97%, 98%, or 99% identical to SEQ ID NO: 1, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 1 or a nucleotide sequence at least 80%, 85%, 90%, 95%, 96%, 97%, 98%, or 99% identical to SEQ ID NO: 1, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 2 or a nucleotide sequence at least 80%, 85%, 90%, 95%, 96%, 97%, 98%, or 99% identical to SEQ ID NO: 2, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 2 or a nucleotide sequence at least 80%, 85%, 90%, 95%, 96%, 97%, 98%, or 99% identical to SEQ ID NO: 2, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 3 or a nucleotide sequence at least 80%, 85%, 90%, 95%, 96%, 97%, 98%, or 99% identical to SEQ ID NO: 3, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 3 or a nucleotide sequence at least 80%, 85%, 90%, 95%, 96%, 97%, 98%, or 99% identical to SEQ ID NO: 3, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 4 or a nucleotide sequence at least 80%, 85%, 90%, 95%, 96%, 97%, 98%, or 99% identical to SEQ ID NO: 4, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12. In some embodiments, the expression construct comprises, in the order of 5′ to 3′, SEQ ID NO: 9, SEQ ID NO: 10, SEQ ID NO: 6, SEQ ID NO: 4 or a nucleotide sequence at least 80%, 85%, 90%, 95%, 96%, 97%, 98%, or 99% identical to SEQ ID NO: 4, and SEQ ID NO: 11, wherein the nucleotide sequence encodes the GLA of SEQ ID NO: 12 or an allelic variant thereof, or a functional GLA polypeptide, such as a GLA polypeptide having at least 10%, 20%, 30%, 40%, 50%, 60% 70%, 80%, 90%, 100% or more activity of the GLA of SEQ ID NO: 12.

In some embodiments, the expression construct comprises, in the order of 5′ to 3′, SEQ ID NO: 9, SEQ ID NO: 10, SEQ ID NO: 5, SEQ ID NO: 1, and SEQ ID NO: 11. In some embodiments, the expression construct comprises, in the order of 5′ to 3′, SEQ ID NO: 9, SEQ ID NO: 10, SEQ ID NO: 6, SEQ ID NO: 1, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 2, and SEQ ID NO: 11. In some embodiments, the expression construct comprises, in the order of 5′ to 3′, SEQ ID NO: 9, SEQ ID NO: 10, SEQ ID NO: 6, SEQ ID NO: 2, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 3, and SEQ ID NO: 11. In some embodiments, the expression construct comprises, in the order of 5′ to 3′, SEQ ID NO: 9, SEQ ID NO: 10, SEQ ID NO: 6, SEQ ID NO: 3, and SEQ ID NO: 11.

In some embodiments, the expression construct comprises, in the order of 5′ to 3′, SEQ ID NO: 9, SEQ ID NO: 10, SEQ ID NO: 5, SEQ ID NO: 4, and SEQ ID NO: 11. In some embodiments, the expression construct comprises, in the order of 5′ to 3′, SEQ ID NO: 9, SEQ ID NO: 10, SEQ ID NO: 6, SEQ ID NO: 4, and SEQ ID NO: 11.

The inventors found that the rAAV of the present invention can achieve a desired safety, e.g., when being administered by intravenous injection. For example, the safety can be evaluated by the comparison of the body weight gain of a subject injected with the rAAV with the body weight gain of a control subject injected with the vehicle for delivering the rAAV Alternatively, the desired safety can be demonstrated by low or even no liver toxicity upon administration by intravenous injection, which can be evaluated, e.g., by ALT and/or AST levels after the injection of the rAAV similar to or same with the control.

The present invention also provides a method for preparing the rAAV. In some embodiments, the rAAV is prepared by a system containing a transgene plasmid comprising the genome of the rAAV, a packaging plasmid encoding the REP and/or CAP proteins, and a helper plasmid, e.g., a host cell such as a mammalian cell comprising the transgene plasmid comprising the genome of the rAAV, the packaging plasmid encoding the REP and/or CAP proteins, and the helper plasmid. Therefore, the present invention also provides a vector such as a plasmid comprising the genome of the rAAV of the invention.

In some embodiments, the rAAV can be packaged as described in Crosson S M et al. [12].

In some embodiments, the genome of the rAAV, from 5′ end of the 5′ ITR to the 3′ end of the 3′ ITR, is about 4,700 nucleotides in length. In some embodiments, the genome of the rAAV, from 5′ end of the 5′ ITR to the 3′ end of the 3′ ITR, is 4,600-4,800, 4,620-4,780, 4,640-4,760, 4,650-4,750, 4,660-4,740, 4,670-4,730, 4,680-4,720 or 4,690-4,710 nucleotides in length. In some embodiments, the genome of the rAAV comprises a stuffer fragment. In some embodiments, the stuffer fragment is downstream of the polyadenylation signal sequence.

In some embodiments, the genome of the rAAV comprises, in the order of 5′ to 3′, 5′ ITR, SEQ ID NO: 9, SEQ ID NO: 10, SEQ ID NO: 5, SEQ ID NO: 4, SEQ ID NO: 11, SEQ ID NO: 26, and 3′ ITR. In some embodiments, the genome of the rAAV comprises, in the order of 5′ to 3′, 5′ ITR, SEQ ID NO: 9, SEQ ID NO: 10, SEQ ID NO: 5, SEQ ID NO: 4, SEQ ID NO: 11, SEQ ID NO: 27, and 3′ ITR.

The present invention also provides a pharmaceutical composition comprising the rAAV Pharmaceutical compositions containing the AAV of the invention can be formulated in any conventional manner by mixing a selected amount of the rAAV with one or more pharmaceutically acceptable carriers or excipients.

Selection of the carrier or excipient is within the skill of the administering professional and can depend upon a number of parameters. These include, for example, the mode of administration (i.e., systemic, oral, local, topical or any other mode) and the disease to be treated. Pharmaceutical carriers or vehicles suitable for administration of the rAAV provided herein include any such carriers known to those skilled in the art to be suitable for the particular mode of administration.

In some embodiments, the pharmaceutical composition is formulated for parenteral administration, e.g., intravenous, intramuscular, or subcutaneous injection. In some embodiments, the pharmaceutical composition is formulated for local administration.

The inventors found that the pharmaceutical composition of the present invention can achieve a desired safety, e.g., when being administered by intravenous injection. For example, the safety can be evaluated by the comparison of the body weight gain of a subject injected with the pharmaceutical composition with the body weight gain of a control subject injected with the vehicle for delivering the rAAV. Alternatively, the desired safety can be demonstrated by low or even no liver toxicity upon administration by intravenous injection, e.g., the liver toxicity can be evaluated by ALT and/or AST levels after the injection of the rAAV similar to or same with the control.

The present invention further provides a vector comprising the genome of the rAAV, wherein the genome of rAAV comprises the expression construct of the invention flanked by 5′ and 3′ ITRs. In some embodiments, the vector is a transgene plasmid for the packaging of rAAV.

The present invention further provides a host cell comprising the polynucleotide, the expression construct, and/or the vector of the present invention. In some embodiment, the host cell is a mammalian cell, such as a human cell. In some embodiment, the host cell is HEK293T cell.

### 4. Treatment of Diseases

The present invention provides a method of treating a disease associated with the deficiency of alpha galactosidase A (α-Gal A), comprising administering the rAAV or the pharmaceutical composition of the present invention to a subject in need thereof. In some embodiments, the disease is Fabry disease. In some embodiments, the rAAV or the pharmaceutical composition is administered by intravenous injection.

The present invention further provides use of the polynucleotide, the expression construct, the rAAV, the pharmaceutical composition, the vector or the host cell of the present invention in the preparation of a medicament for treating a disease associated with the deficiency of alpha galactosidase A (α-Gal A) in a subject in need thereof. In some embodiments, the disease is Fabry disease. In some embodiments, the medicament is administered by intravenous injection.

Provided is the rAAV and/or the pharmaceutical composition of the present invention, for use in the treatment of a disease associated with the deficiency of alpha galactosidase A (α-Gal A) in a subject in need thereof. In some embodiments, the disease is Fabry disease. In some embodiments, the rAAV and/or the pharmaceutical composition is administered by intravenous injection.

In some embodiments, the treatment increases the GLA activity in the subject. In some embodiments, the treatment increases the GLA activity in the tissues including but not limited to plasma, kidney, liver, heart, brain and spinal cord.

In some embodiments, the treatment decreases the Gb3 and/or lyso-Gb3 concentrations in the subject. In some embodiments, the treatment decreases the Gb3 and/or lyso-Gb3 concentrations in the tissues including but not limited to plasma, kidney, liver, heart, brain and spinal cord.

In some embodiments, the subject is a human. In some embodiments, the subject is male. In some embodiments, the subject is female.

In some embodiments, the treatment cures, improves, alleviates, blocks or partially blocks the symptoms of Fabry disease.

### EXAMPLES

The following Examples are provided for illustration only, rather than for any limitation to the present application.

### Example 1. Preparation of the rAAVs

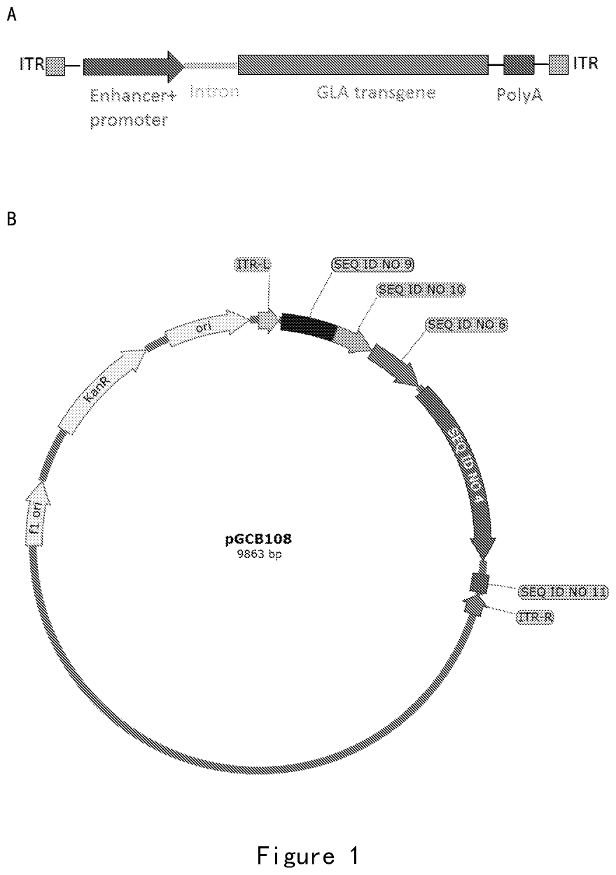

Seven constructs comprising the genomes of rAAVs were prepared by Gibson Assembling the pGCB108 plasmid comprising the cytomegalovirus (CMV) early enhancer (SEQ ID NO: 9) and the chicken beta-actin core promoter (SEQ ID NO: 10) with the introns and coding sequences (various versions of codon optimization) shown in Table 1. The structure of the constructs is shown in FIG. 1A.

TABLE 1
Elements in the constructs

| | Enhancer and | | hGLA Coding | |
|---|---|---|---|---|
| | Promoter | Intron | Sequence | PolyA |
| | (SEQ | (SEQ | (SEQ | (SEQ |
| Constructs | ID NO:) | ID NO:) | ID NO:) | ID NO:) |
| 103 | 9 and 10 | 5 | 1 | 11 |
| 104 | 9 and 10 | 5 | 2 | 11 |
| 105 | 9 and 10 | 5 | 3 | 11 |
| 106 | 9 and 10 | 5 | 4 | 11 |
| 108 | 9 and 10 | 6 | 4 | 11 |
| 109 | 9 and 10 | 7 | 4 | 11 |
| 110 | 9 and 10 | 8 | 4 | 11 |

As an example, and the map of the transgene plasmid comprising construct 108 is shown in FIG. 1B, and the sequence thereof is shown in SEQ ID NO: 13.

The rAAVs were produced by packaging the genomes in hAAV9 capsid as described in Crosson S M et al. [12]. Briefly, HEK293T cells were cotransfected, using polyethylenimine, with the helper plasmid, the packaging plasmid encoding rep2/cap9 and the transgene plasmid. Following 72 h incubation at 37° C., cells were harvested, and viral particles were purified through an iodixanol gradient. Virus titer, expressed as vg per milliliter, was measured by qPCR using a pair of primers (SEQ ID NOs: 14 and 15) and a probe (SEQ ID NO: 16, with 5′ FAM and 3′ BHQ-1 modifications) targeting RBG polyA. A Pvu I linearized transgene plasmid was prepared to generate a standard curve. The thermal cycling conditions were 95° C. for 10 min, followed by 40 cycles of 95° C. for 15 s and 60° C. for 1 min.

The titers of the obtained rAAVs were shown in Table 2, in which the rAAVs were named with the constructs comprised therein, e.g., rAAV 106 means the rAAV comprising construct 106 in Table 1. It can be seen that the rAAVs were obtained at comparable levels.

TABLE 2
Titers of the obtained rAAVs

| rAAVs | Titer (vg/mL) |
|---|---|
| 103 | 3.60E+13 |
| 104 | 1.91E+13 |
| 105 | 2.76E+13 |
| 106 | 3.10E+13 |
| 108 | 6.32E+13 |
| 109 | 8.51E+13 |
| 110 | 6.70E+13 |

### Example 2. Expression of GLA in Wild-Type Mice with the rAAVs

The seven rAAVs prepared in Example 1 were diluted in formulation buffer (DPBS) and injected into the tail veins of 6- to 8-week-old wild-type C57BL/6 male mice at the dose of 5e10 vg, 1.6e11 vg or 5e11 vg per mouse.

Mice were sacrificed 1 month after the injection of the rAAVs. Blood samples were collected in EDTA K2-coated tubes from medial canthus of the eye, and plasma was isolated by centrifugation at 2,000 g for 15 min at 4° C. The tissues were snap frozen in liquid nitrogen and stored at −80° C. until use.

The GLA activity in plasma and the tissue samples were analyzed using the BioVision alpha galactosidase (α-Gal) activity assay kit according to the manufacturer's instructions.

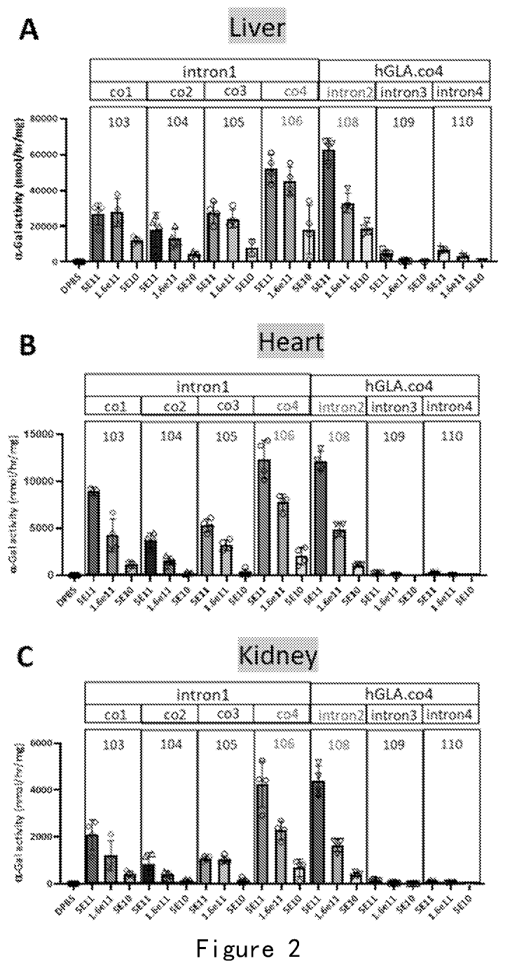

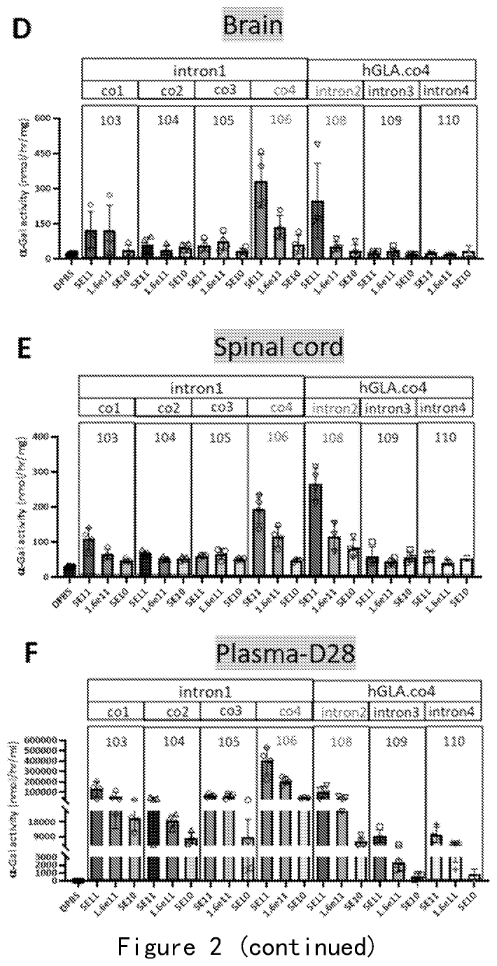

As shown in FIG. 2, the GLA activity was dose-dependent; rAAVs 106 and 108 resulted in the highest activities of α-Gal A in all of the tested samples (FIGS. 2A-2F), indicating that SEQ ID NO: 4 surprisingly led to a higher expression of GLA than SEQ ID NOs: 1, 2 and 3. The rAAV 103 resulted in higher GLA activities than rAAVs 104 and 105 in heart (FIG. 2B), kidney (FIG. 2C), brain (FIG. 2D), spinal cord (FIG. 2E) and plasma (FIG. 2F); and rAAVs 103 and 105 resulted in higher GLA activities than rAAV 104 in liver (FIG. 2A), heart (FIG. 2B), kidney (FIG. 2C) and plasma (FIG. 2F).

The higher level of GLA activities achieved by rAAVs 106 and 108 than rAAVs 109 and 110 (FIGS. 2A-2F) further indicated that the introduction of an intron of SEQ ID NO: 5 or 6, i.e., a longer intron, (e.g., longer than 400 bp, 1065 bp for rAAV 106, and 407 bp for rAAV 108) resulted in much higher expression of GLA than a short intron (133 bp for rAAV 109, and 157 bp for rAAV 110).

### Example 3. Treatment of Fabry Disease Model (GLA KO Mice) with rAAVs

3.1. Evaluation of the Short-Term Efficacy of the Treatments with rAAVs 106 and 108 in GLA KO Mice

The rAAVs 106 and 108 were diluted in formulation buffer (DPBS) and injected into the tail veins of 3-month-old female and male GLA KO mice (Purchased from The Jackson Laboratory, https://www.jax.org/strain/003535) at the dose of 5e10 vg or 5e11 vg per mouse. WT male, HE (one of the GLA copies was mutated) female and GLA KO mice injected with DPBS were used as controls.

Mice were sacrificed 1 month after the injection of the rAAVs. Blood samples were collected in EDTA K2-coated tubes from medial canthus of the eye, and plasma was isolated by centrifugation at 2,000 g for 15 min at 4° C. The tissues were snap frozen in liquid nitrogen and stored at −80° C. until use.

The GLA activity of in plasma and the tissue samples were analyzed using the BioVision alpha galactosidase (α-Gal) activity assay kit according to the manufacturer's instructions. The Gb3 concentrations in plasma and kidney were determined using liquid chromatography with tandem mass spectrometry (LC-MS/MS) at Chempartner.

In particular, tissues were homogenized in 5-fold water. An aliquot of 15 μL tissue homogenates or plasma was diluted with 30 μl water, and the mixture was vortexed for 1 min. 45 μL diluted plasma or tissue homogenates was mixed with 25 μL internal standard (N-C18:0-CD3-GB3 (IS), 1000 ng/mL) in DMSO, and then mixed with 1 mL chloroform/methanol (2:1, v/v). The mixture was vortexed for 10 min, and centrifuged at 14000 rpm for 5 min. 750 μL supernatant was transferred to a new tube, and dried under N₂. The sample was reconstituted with 100 μL DMSO, and then mixed with 100 μL MeOH. The mixture was vortexed for 2 min, and centrifuged at 5800 rpm for 10 min. 60 μL supernatant was transferred to a new plate. 3 μl of the supernatant was injected to LC-MS/MS. The LC system comprised a Shimadzu (Shimadzu Co., Japan) liquid chromatography equipped with a binary pump (LC-30AD), an autosampler (SIL-30AC), a column oven (CTO-20A), a system controller (CBM-20A) and a degasser (DGU-20A). Mass spectrometric analysis was performed using an AB SCIEX API6500+ triple-quadrupole (Ontario, Canada) instrument with an ESI interface. The data acquisition and control system were created by using Analyst 1.6.3 software from AB SCIEX (Ontario, Canada). Chromatographic separation on a Waters BEH C18 Column (2.1*50 mm, 1.7 μm), mobile phase A is H₂O-acetonitrile (ACN) (95:5)-0.1% Fomic acid (FA), mobile phase B is ACN-isopropanol (IPA)-MeOH(40:40:20)-0.1% FA. The column was eluted at a flow rate of 0.5 mL/min in a gradient program consisting of 70% phase B(0-0.2 min), from 70 to 100% B(0.2-1.0 min), 100% B (1.00-3.00 min), from 100 to 70% B (3.00-3.10 min), 70% B (3.10-4.00 min). For Gb3, the retention time for the analyte and IS (N-C18:0-CD3-GB3) were Gb3(d18:1)(C16:0) 1.60 min, Gb3(d18:1)(C18:0) 1.69 min, Gb3(d18:1)(C20:0) 1.79 min, Gb3(d18:1)(C22:0) 1.91 min, Gb3(d18:1)(C24:1) 1.89 min, Gb3(d18:1)(C24:0) 2.05 min and N-C18:0-CD3-GB3(IS) 1.69 min, respectively. The injection volume is 3 μL. The precursor product ion pair was m/z 1046.7→884.6 for Gb3(d18:1)(C16:0), m/z 1074.7→912.6 for Gb3(d18:1)(C18:0), m/z 1102.7→940.6 for Gb3(d18:1)(C20:0), m/z 1130.7→968.6 for Gb3(d18:1)(C22:0), m/z 1156.7→994.6 for Gb3(d18:1)(C24:1), m/z 1158.7→996.6 for Gb3(d18:1)(C24:0), m/z 1077.8→915.6 for N-C18:0-CD3-GB3.

The samples were IHC stained for GLA (liver, heart, brain and spinal cord tissues) and Gb3 (liver tissues).

hGLA IHC was performed using an automatic protocol developed for the detection of human GLA protein. Briefly, formalin-fixed paraffin embedded (FFPE) tissue samples were sectioned, and de-paraffinized and rehydrated. Antigen retrieval was performed with Bond Epitope Retrieval Solution (ER1) pH6.0 at 100° C. for 20 min (AR9961, Leica) prior to immunohistochemical analysis. Specific detection of human hGLA protein was achieved using a human-specific anti-GLA rabbit polyclonal antibody (HPA000237, Sigma).

Visualization under light microscopy was achieved following incubation with Anti-rabbit Poly-HRP-immunoglobulin G (IgG) secondary antibody for subsequent reaction with 3,3′-diaminobenzidene (DAB) substrate (DS9800, Leica, containing both the Anti-rabbit Poly-HRP-immunoglobulin G (IgG) and DAB substrate), resulting in the deposition of brown stain. The slides were counterstained with hematoxylin and the images were captured using an Aperio ScanScope for whole slide.

Gb3 IHC was performed using a manual protocol developed for the detection of GB3 protein. Briefly, the tissues were fixed in 4% paraformaldehyde, cryoprotected by infiltration with 30% sucrose, and frozen in freezing medium (Tissue-Tek OCT compound, SAKURA, #4583). The frozen tissues were sectioned and the sections (10 μm) were rinsed in PBS and then immersed in 0.3% hydrogen peroxide (in PBS) for 30 min at room temperature. Specific detection of Gb3 protein was achieved using goat anti-mouse IgG Fab fragment (115-007-003, Jackson IR) followed by the incubation with anti-Gb3 mouse monoclonal antibody (BGR23, AMS.A2506, Amsbio). Visualization under light microscopy was achieved following incubation with mouse-on-mouse HRP—polymer secondary antibody (MM620, Biocare) for subsequent reaction with 3, 3′-diaminobenzidene (DAB) substrate (BDB2004, Biocare), resulting in the deposition of brown stain. The slides were counterstained with hematoxylin and the images were captured using an Aperio ScanScope for whole slide.

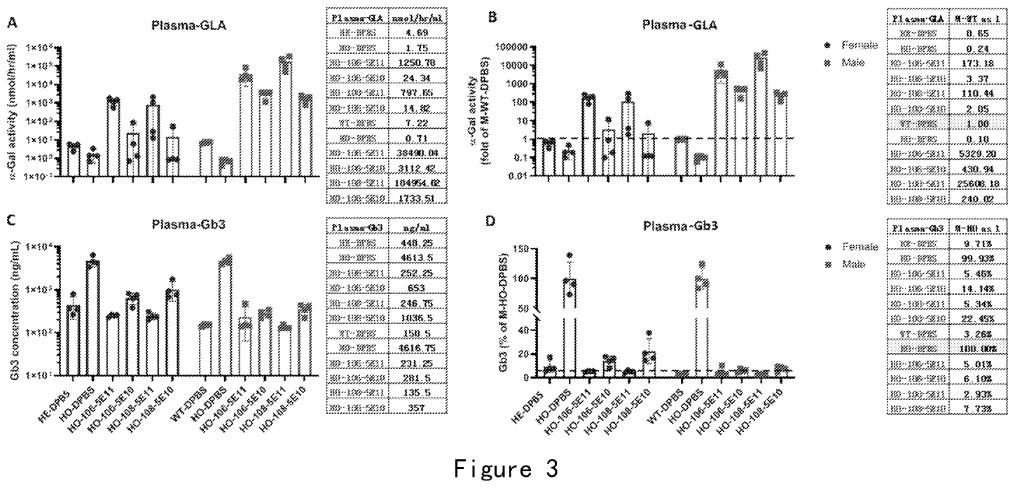

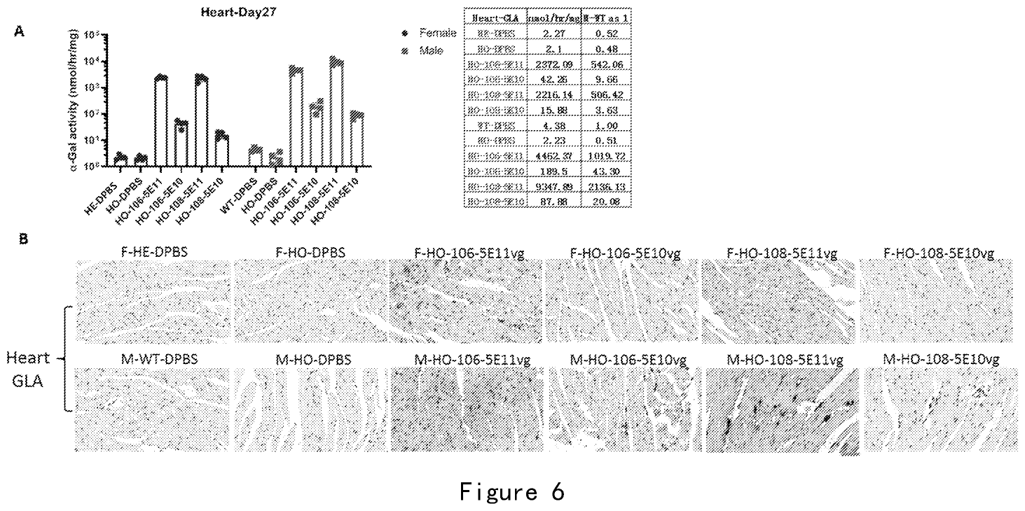

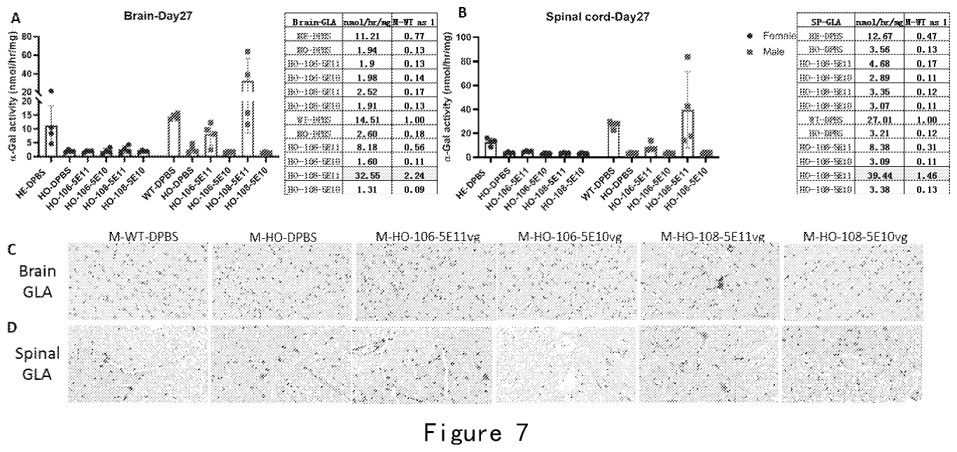

It was shown that the GLA activities in the tissues of GLA KO mice were significantly increased in plasma, kidney, liver and heart by the administration of rAAVs in a dose-dependent manner, i.e., the administration of rAAV at a higher dose (5e11 vg) resulted in a higher GLA activity (FIGS. 3A, 3B, 4A, 4B, 5A, 5B, 6A and 6B). The GLA activities in brain and spinal cord showed difference between female and male mice. In particular, the GLA activities in female GLA KO mice administered with rAAVs were not significantly increased in brain and spinal cord, while the administration of rAAVs at a high dose increased the GLA activity in male GLA KO mice (FIG. 7).

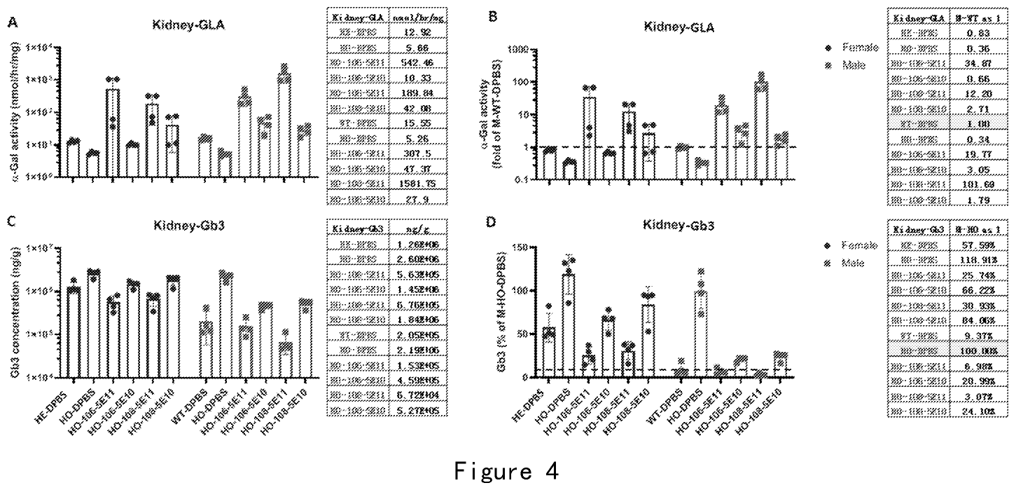

The Gb3 concentrations in plasma and kidney were significantly higher in the GLA KO mice injected with DPBS than the WT and HE mice; the Gb3 concentrations were significantly decreased by the administration of rAAVs; and the Gb3 concentrations in GLA KO mice treated with high dose of rAAVs were similar to that in the WT mice (FIGS. 3D and 4D).

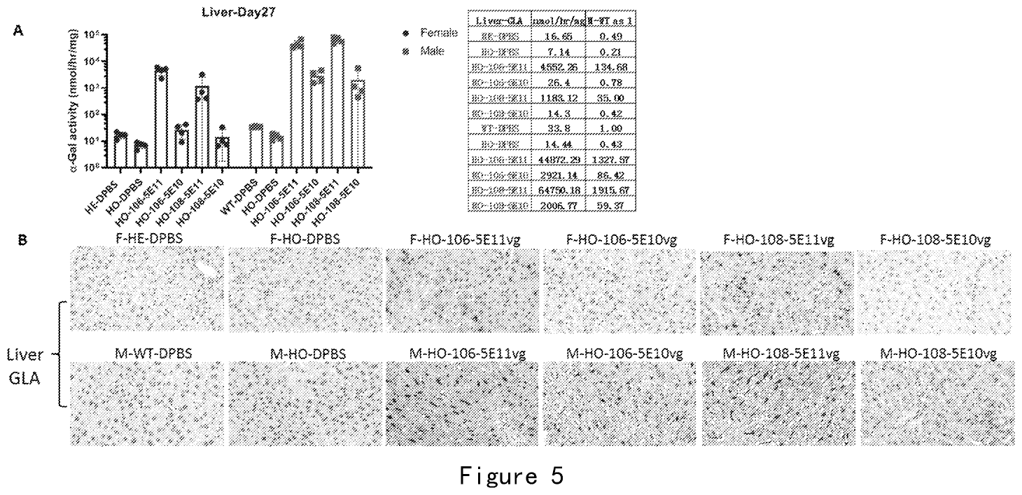

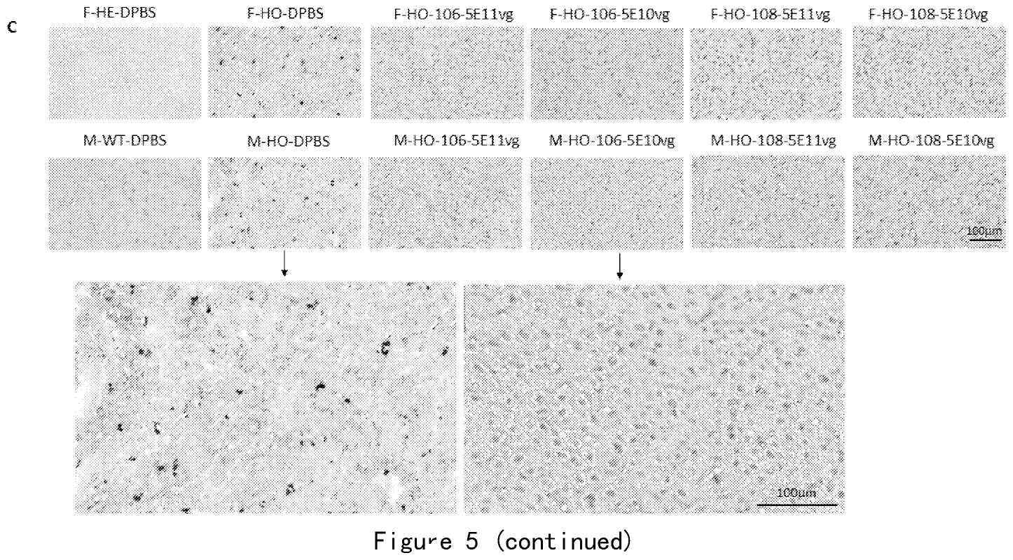

The administration of rAAVs also resulted in the clearance of Gb3 in liver (FIG. 5C).

The GLA activities and Gb3 concentrations in various samples are detailed in Tables 2 to 9 (“HO” represents homozygous knockout mouse).

TABLE 2
GLA activities in plasma.

| | | GLA activity | Activity relative |
|---|---|---|---|
| Mice | Treatment | (nmol/hr/ml) | to WT male |
| HE-female | DPBS | 4.69 | 0.65 |
| HO-female | DPBS | 1.75 | 0.24 |
| HO-female | rAAV 106 5E11 | 1250.78 | 173.18 |
| HO-female | rAAV 106 5E10 | 24.34 | 3.37 |
| HO-female | rAAV 108 5E11 | 797.65 | 110.44 |
| HO-female | rAAV 108 5E10 | 14.82 | 2.05 |
| WT-male | DPBS | 7.22 | 1.00 |
| HO-male | DPBS | 0.71 | 0.10 |
| HO-male | rAAV 106 5E11 | 38490.04 | 5329.20 |
| HO-male | rAAV 106 5E10 | 3112.42 | 430.94 |
| HO-male | rAAV 108 5E11 | 184954.62 | 25608.18 |
| HO-male | rAAV 108 5E10 | 1733.51 | 240.02 |

TABLE 3
Gb3 concentration in plasma.

| | | Gb3 | Percent of the Gb3 |
|---|---|---|---|
| | | concentration | concentration in HO |
| Mice | Treatment | (ng/ml) | male control |
| HE-female | DPBS | 448.25 | 9.71% |
| HO-female | DPBS | 4613.5 | 99.93% |
| HO-female | rAAV 106 5E11 | 252.25 | 5.46% |
| HO-female | rAAV 106 5E10 | 653 | 14.14% |
| HO-female | rAAV 108 5E11 | 246.75 | 5.34% |
| HO-female | rAAV 108 5E10 | 1036.5 | 22.45% |
| WT-male | DPBS | 150.5 | 3.26% |
| HO-male | DPBS | 4616.75 | 100.00% |
| HO-male | rAAV 106 5E11 | 231.25 | 5.01% |
| HO-male | rAAV 106 5E10 | 281.5 | 6.10% |
| HO-male | rAAV 108 5E11 | 135.5 | 2.93% |
| HO-male | rAAV 108 5E10 | 357 | 7.73% |

TABLE 4
GLA activity in kidney.

| | | GLA activity | Activity relative |
|---|---|---|---|
| Mice | Treatment | (nmol/hr/mg) | to WT male |
| HE-female | DPBS | 12.92 | 0.83 |
| HO-female | DPBS | 5.66 | 0.36 |
| HO-female | rAAV 106 5E11 | 542.46 | 34.87 |
| HO-female | rAAV 106 5E10 | 10.33 | 0.66 |
| HO-female | rAAV 108 5E11 | 189.84 | 12.20 |
| HO-female | rAAV 108 5E10 | 42.08 | 2.71 |
| WT-male | DPBS | 15.55 | 1.00 |
| HO-male | DPBS | 5.26 | 0.34 |
| HO-male | rAAV 106 5E11 | 307.5 | 19.77 |
| HO-male | rAAV 106 5E10 | 47.37 | 3.05 |
| HO-male | rAAV 108 5E11 | 1581.75 | 101.69 |
| HO-male | rAAV 108 5E10 | 27.9 | 1.79 |

TABLE 5
Gb3 concentration in kidney.

| | | Gb3 | Percent of the Gb3 |
|---|---|---|---|
| | | concentration | concentration in HO |
| Mice | Treatment | (ng/g) | male control |
| HE-female | DPBS | 1.26E+06 | 57.59% |
| HO-female | DPBS | 2.60E+06 | 118.91% |
| HO-female | rAAV 106 5E11 | 5.63E+05 | 25.74% |
| HO-female | rAAV 106 5E10 | 1.45E+06 | 66.22% |
| HO-female | rAAV 108 5E11 | 6.76E+05 | 30.93% |
| HO-female | rAAV 108 5E10 | 1.84E+06 | 84.06% |
| WT-male | DPBS | 2.05E+05 | 9.37% |
| HO-male | DPBS | 2.19E+06 | 100.00% |
| HO-male | rAAV 106 5E11 | 1.53E+05 | 6.98% |
| HO-male | rAAV 106 5E10 | 4.59E+05 | 20.99% |
| HO-male | rAAV 108 5E11 | 6.72E+04 | 3.07% |
| HO-male | rAAV 108 5E10 | 5.27E+05 | 24.10% |

TABLE 6
GLA activity in liver.

| | | GLA activity | Activity relative |
|---|---|---|---|
| Mice | Treatment | (nmol/hr/mg) | to WT male |
| HE-female | DPBS | 16.65 | 0.49 |
| HO-female | DPBS | 7.14 | 0.21 |
| HO-female | rAAV 106 5E11 | 4552.26 | 134.68 |
| HO-female | rAAV 106 5E10 | 26.4 | 0.78 |
| HO-female | rAAV 108 5E11 | 1183.12 | 35.00 |
| HO-female | rAAV 108 5E10 | 14.3 | 0.42 |
| WT-male | DPBS | 33.8 | 1.00 |
| HO-male | DPBS | 14.44 | 0.43 |
| HO-male | rAAV 106 5E11 | 44872.29 | 1327.57 |
| HO-male | rAAV 106 5E10 | 2921.14 | 86.42 |
| HO-male | rAAV 108 5E11 | 64750.18 | 1915.67 |
| HO-male | rAAV 108 5E10 | 2006.77 | 59.37 |

TABLE 7
GLA activity in heart.

| | | GLA activity | Activity relative |
|---|---|---|---|
| Mice | Treatment | (nmol/hr/mg) | to WT male |
| HE-female | DPBS | 2.27 | 0.52 |
| HO-female | DPBS | 2.1 | 0.48 |
| HO-female | rAAV 106 5E11 | 2372.09 | 542.06 |
| HO-female | rAAV 106 5E10 | 42.26 | 9.66 |
| HO-female | rAAV 108 5E11 | 2216.14 | 506.42 |
| HO-female | rAAV 108 5E10 | 15.88 | 3.63 |
| WT-male | DPBS | 4.38 | 1.00 |
| HO-male | DPBS | 2.23 | 0.51 |
| HO-male | rAAV 106 5E11 | 4462.37 | 1019.72 |
| HO-male | rAAV 106 5E10 | 189.5 | 43.30 |
| HO-male | rAAV 108 5E11 | 9347.89 | 2136.13 |
| HO-male | rAAV 108 5E10 | 87.88 | 20.08 |

TABLE 8
GLA activity in brain.

| | | GLA activity | Activity relative |
|---|---|---|---|
| Mice | Treatment | (nmol/hr/mg) | to WT male |
| HE-female | DPBS | 11.21 | 0.77 |
| HO-female | DPBS | 1.94 | 0.13 |
| HO-female | rAAV 106 5E11 | 1.9 | 0.13 |
| HO-female | rAAV 106 5E10 | 1.98 | 0.14 |
| HO-female | rAAV 108 5E11 | 2.52 | 0.17 |
| HO-female | rAAV 108 5E10 | 1.91 | 0.13 |
| WT-male | DPBS | 14.51 | 1.00 |
| HO-male | DPBS | 2.60 | 0.18 |
| HO-male | rAAV 106 5E11 | 8.18 | 0.56 |
| HO-male | rAAV 106 5E10 | 1.60 | 0.11 |
| HO-male | rAAV 108 5E11 | 32.55 | 2.24 |
| HO-male | rAAV 108 5E10 | 1.31 | 0.09 |

TABLE 9
GLA activity in spinal cord.

| | | GLA activity | Activity relative |
|---|---|---|---|
| Mice | Treatment | (nmol/hr/mg) | to WT male |
| HE-female | DPBS | 12.67 | 0.47 |
| HO-female | DPBS | 3.56 | 0.13 |
| HO-female | rAAV 106 5E11 | 4.68 | 0.17 |
| HO-female | rAAV 106 5E10 | 2.89 | 0.11 |
| HO-female | rAAV 108 5E11 | 3.35 | 0.12 |
| HO-female | rAAV 108 5E10 | 3.07 | 0.11 |
| WT-male | DPBS | 27.01 | 1.00 |
| HO-male | DPBS | 3.21 | 0.12 |
| HO-male | rAAV 106 5E11 | 8.38 | 0.31 |
| HO-male | rAAV 106 5E10 | 3.09 | 0.11 |
| HO-male | rAAV 108 5E11 | 39.44 | 1.46 |
| HO-male | rAAV 108 5E10 | 3.38 | 0.13 |

3.2. Comparison of the Short-Term Efficacy of the Treatments with rAAVs 108, 109 and 110 in GLA KO Mice

The rAAVs 108, 109 and 110 were diluted in formulation buffer (DPBS) and injected into the tail veins of 3-month-old male WT and GLA KO mice. Male WT and GLA KO mice injected with DPBS were used as controls. The treatments are detailed in Table 10.

TABLE 10

| | | | Dose | Number | i.v. |
|---|---|---|---|---|---|
| Mice | | Dose | (vg/20 g | of | Injection |
| type | Treatment | (vg/kg) | mouse) | animals | (μl/mouse) |
| M-WT | DPBS + 0.001% | 0 | 0 | 5 | 200 |
| | Pluronic ™ F-68 | | | | |
| | (PF68) | | | | |
| M-KO | DPBS + 0.001% | 0 | 0 | 5 | 200 |
| | PF68 | | | | |
| M-KO | rAAV 108 | 1.25E+13 | 2.50E+11 | 5 | Depends |
| | | | | | on BW |
| M-KO | rAAV 109 | 1.25E+13 | 2.50E+11 | 5 | Depends |
| | | | | | on BW |
| M-KO | rAAV 110 | 1.25E+13 | 2.50E+11 | 5 | Depends |
| | | | | | on BW |

Plasma was sampled as described in Example 3.1 four weeks after the injection, and mice were sacrificed 1 month after the injection of the rAAVs. The tissues were snap frozen in liquid nitrogen and stored at −80° C. until use.

The genome copies of the rAAVs were measured by qPCR specific for RGB polyA, and the hGLA mRNA levels were also measured by qPCR. The qPCR was performed using the TaqMan® Universal Master Mix II, with UNG (Thermo Scientific) and QuantStudio 5 Real-Time PCR System, following the procedures recommended by the manufacturer.

For the quantification of the genome copies of rAAVs, total DNA was isolated from tissue samples using Omega E.Z.N.A.® DNA/RNA Kit. The qPCR targeting RGB polyA was performed with a pair of primers (SEQ ID NOs: 14 and 15) and a probe (SEQ ID NO: 16, with 5′ FAM and 3′ BHQ-1 modifications). The mice genomic DNA was quantified in parallel samples using a pair of primers (SEQ ID NOs: 17 and 18) and a probe (SEQ ID NO: 19, with 5′ HEX and 3′ BHQ-1 modifications) specific for murine beta-actin. The thermal cycling conditions were 95° C. for 10 min, followed by 40 cycles of 95° C. for 15 s and 60° C. for 1 min. The standards were 10-fold serial dilutions of transgene plasmid for AAV vector DNA measurements and 10-fold serial dilutions of mouse beta actin DNA as the endogenous reference.

For the measurement of hGLA mRNA levels, total RNA was extracted from tissue samples using Omega E.Z.N.A.® DNA/RNA Kit and was then reverse-transcribed using Applied Biosystems™ High-Capacity RNA-to-cDNA™ Kit. The qPCR was performed with cDNA as the template, a pair of primers (SEQ ID NOs: 20 and 21) and a probe (SEQ ID NO: 22, with 5′ FAM and 3′ BHQ-1 modifications) specific for hGLA, and a pair of primers (SEQ ID NOs: 23 and 24) and a probe (SEQ ID NO: 25, with 5′ HEX and 3′ BHQ-1 modifications) specific for the murine GAPDH gene. The thermal cycling conditions were 95° C. for 10 min, followed by 40 cycles of 95° C. for 15 s and 60° C. for 1 min. The standards were 10-fold serial dilutions of transgene plasmid for transgene measurements and 10-fold serial dilutions of mouse GAPDH cDNA as the endogenous reference.

The GLA activity of in plasma and the tissue samples were analyzed using the BioVision alpha galactosidase (α-Gal) activity assay kit according to the manufacturer's instructions. The Gb3 concentrations in plasma and kidney were determined using LC-MS/MAS as described in Example 3.1. The tissues were IHC stained for GLA and Gb3 as described in Example 3.1.

As shown in FIG. 8, the GLA activities in the tissues and plasma from mice injected with rAAV 108 was much higher than those from mice injected with rAAVs 109 and 110.

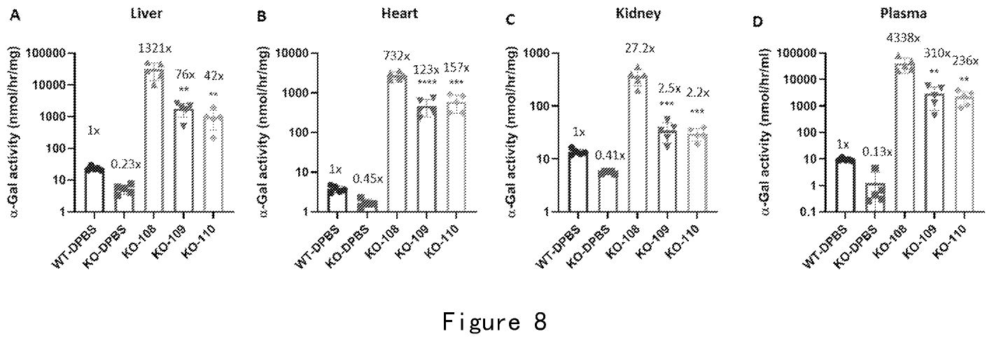

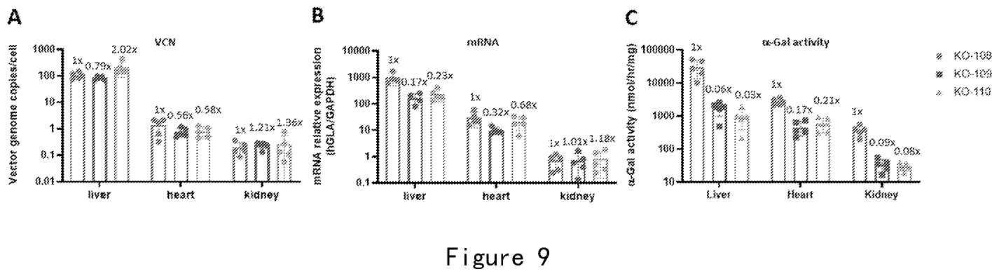

As shown in FIG. 9, the transcription and translation levels of GLA were much higher in the mice injected with rAAV 108 than rAAVs 109 and 110. In particular, the genome copies of rAAVs 109 and 110 in the tissues were similar to or even higher than the genome copies of rAAV 108, while the mRNA levels were much lower, and the ratio between the GLA activities were even lower than the ratio between the mRNA levels, indicating that both the transcription and the translation were much higher with rAAV 108.

As shown in FIG. 10, the Gb3 levels in kidney and plasma from mice injected with rAAV 108 were significantly lower than rAAVs 109 and 110.

It can be seen that in liver, heart and plasma, the GLA expressed by rAAVs 109 and 110 may be enough to reduce the Gb3 to low levels; but in kidney, rAAVs 109 and 110 could not provide as much GLA as rAAV 108 to reduce Gb3 to a desired low level.

### Example 4. Evaluation of the Long-Term Efficacy of the Treatments with rAAVs 106 and 108 in GLA KO Mice

The rAAVs 106 and 108 were diluted in formulation buffer and injected into the tail veins of 5-month-old female and male GLA KO mice (HO) at high dose (HD) of 2.5E13 vg/kg or low dose (LD) of 2.5E12 vg/kg. WT male, HE (one of the GLA copies was mutated) female and GLA KO mice injected with DPBS were used as controls. The treatments are detailed in Table 11.

TABLE 11

| | Mice | | Dose | Dose | Number of | i.v. Injection |
|---|---|---|---|---|---|---|
| Group | type | Treatment | (vg/kg) | (vg/mouse) | Animals | (μl/mouse) |
| G1 | F-HE | DPBS + 0.001% PF68 | 0 | 0 | 6 | 200 |
| G2 | F-HO | DPBS + 0.001% PF68 | 0 | 0 | 6 | 200 |
| G3 | F-HO | rAAV 106 | 2.50E+13 | 5.00E+11 | 7 | 200 |
| G4 | F-HO | rAAV 106 | 2.50E+12 | 5.00E+10 | 7 | 200 |
| G5 | F-HO | rAAV 108 | 2.50E+13 | 5.00E+11 | 7 | 200 |
| G6 | F-HO | rAAV 108 | 2.50E+12 | 5.00E+10 | 7 | 200 |
| G7 | M-WT | DPBS + 0.001% PF68 | 0 | 0 | 7 | 200 |
| G8 | M-HO | DPBS + 0.001% PF68 | 0 | 0 | 6 | 200 |
| G9 | M-HO | rAAV 106 | 2.50E+13 | 5.00E+11 | 7 | 200 |
| G10 | M-HO | rAAV 106 | 2.50E+12 | 5.00E+10 | 7 | 200 |
| G11 | M-HO | rAAV 108 | 2.50E+13 | 5.00E+11 | 7 | 200 |
| G12 | M-HO | rAAV 108 | 2.50E+12 | 5.00E+10 | 6 | 200 |

Blood samples were collected in EDTA K2-coated tubes from medial canthus of the eye at 2, 4, 6, 8, 12, and 16 weeks after AAV injection, and plasma was isolated by centrifugation at 2,000 g for 15 min at 4° C. Mice were sacrificed at 24 weeks (for female) or 26 weeks (for male) after the rAAV injection and tissues were snap frozen in liquid nitrogen and stored at −80° C. until use. The GLA activities in tissue samples and plasma were analyzed using the BioVision alpha galactosidase (α-Gal) activity assay kit according to the manufacturer's instructions. Gb3 concentrations in tissues and plasma were determined using liquid chromatography with tandem mass spectrometry (LC-MS/MS) at Chempartner as described in Example 3.1.

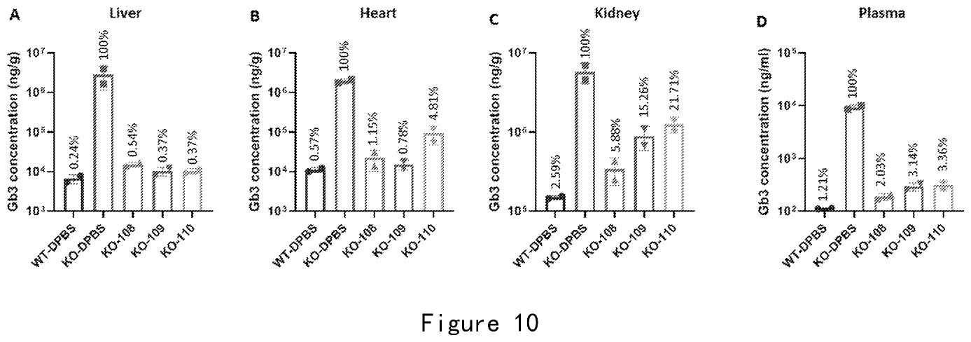

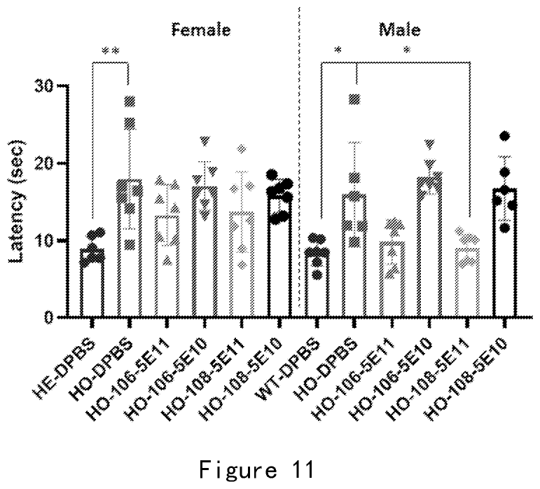

The nociceptive response to heat stimulus was measured using a hot-plate test at 22 weeks after the rAAV injection. The mice were individually placed on a 55° C. surface, and the time taken to respond with a characteristic shake or flick of the hind paw was recorded as the latency period. If no response was evident by 60 seconds, the mice were removed from the hot plate to prevent thermal injury. As shown in FIG. 11, the treatment with high dose of rAAV 106 or 108 reduced the latency periods in GLA KO mice in hot plate test, and the reduction for male mice was more significant (t-test: compared with HO-DPBS, * p<0.05, **p<0.01).

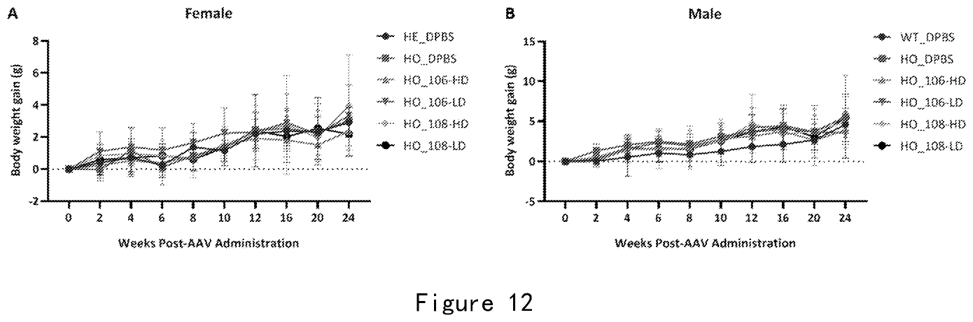

Body weights were measured bi-weekly from day 0 through 24 weeks post-injection (p.i.). Body weight gain was calculated as the difference between each of the time points and day 0. As shown in FIG. 12, the body weight gains were not significantly different between the groups, indicating the desired safety of rAAVs 106 and 108.

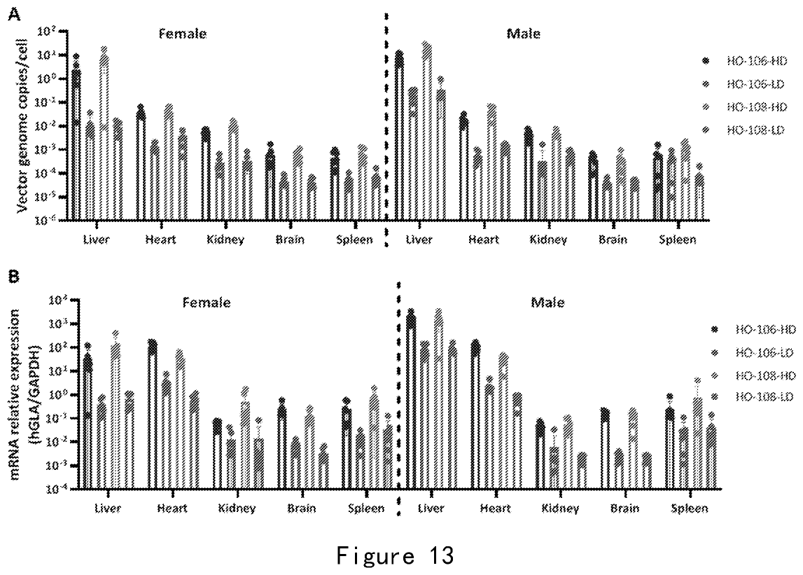

The tissues were assayed by qPCR at 24 weeks (female) or 26 weeks (male) p.i. for vector genome to determine the biodistribution of the rAAVs in GLA KO mice following a systemic delivery. Further, RNA was extracted from different tissues at 24 weeks (female) or 26 weeks (male) p.i., and mRNA levels for the hGLA transgene were determined. The qPCR was performed as described in Example 3.2. As shown in FIG. 13A, the biodistributions of rAAV 106 and rAAV 108 were similar, which were both in a dose-dependent manner, and the levels in liver was the highest. The mRNA levels matched the DNA levels (FIG. 13B).

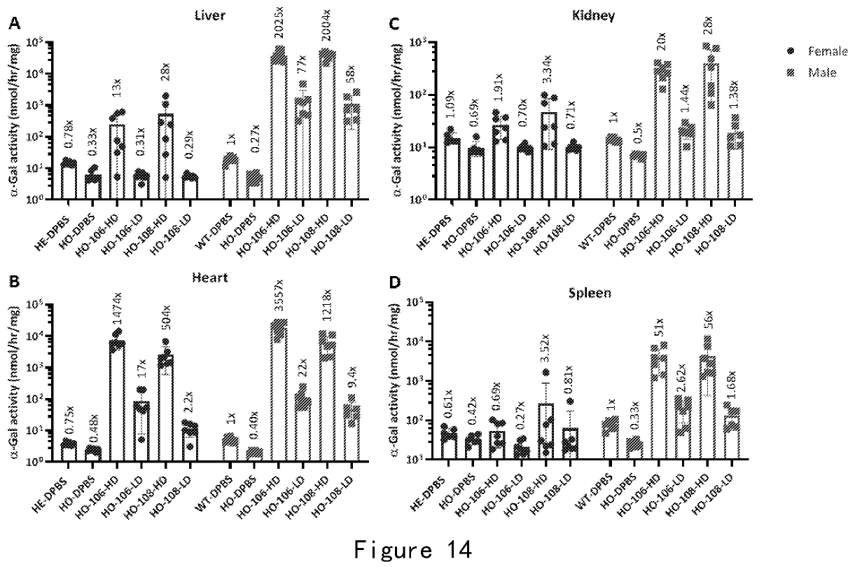

The GLA activities in tissue samples were detected as described previously. As shown in FIG. 14, the GLA activities in the tissues were increased by the injection of rAAVs 106 and 108 in a dose-dependent manner in both female and male GLA KO mice, while the levels in male tissues were higher than female tissues, including liver (FIG. 14A), heart (FIG. 14B), kidney (FIG. 14C), and spleen (FIG. 14D).

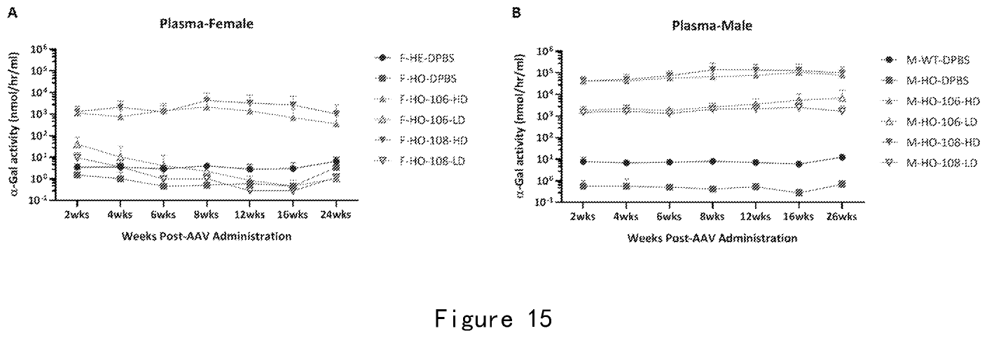

The GLA activities in plasma were shown in FIG. 15. The GLA activities in male plasma were stable over six months, while the GLA activities in the plasma from females with LD injection were decreased from 2 to 16 weeks p.i., and the GLA activities in the plasma from females with HD injection were stable.

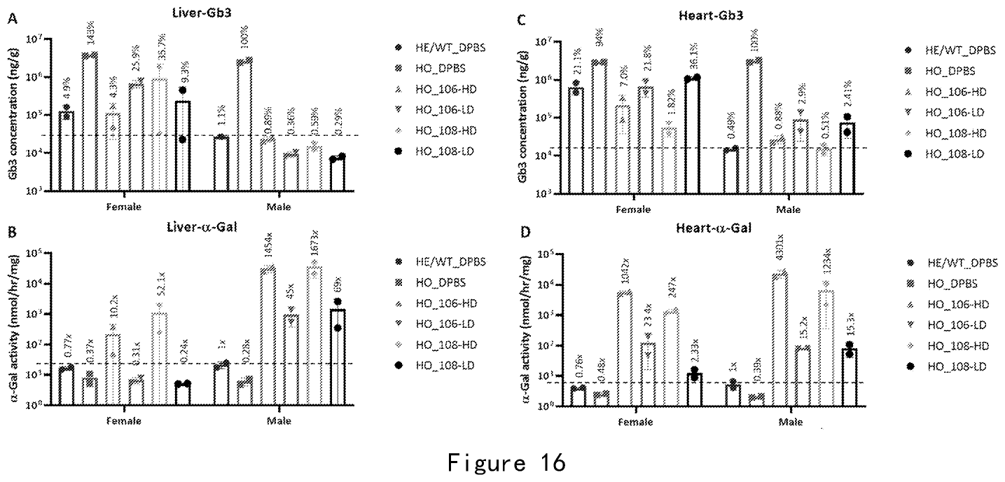

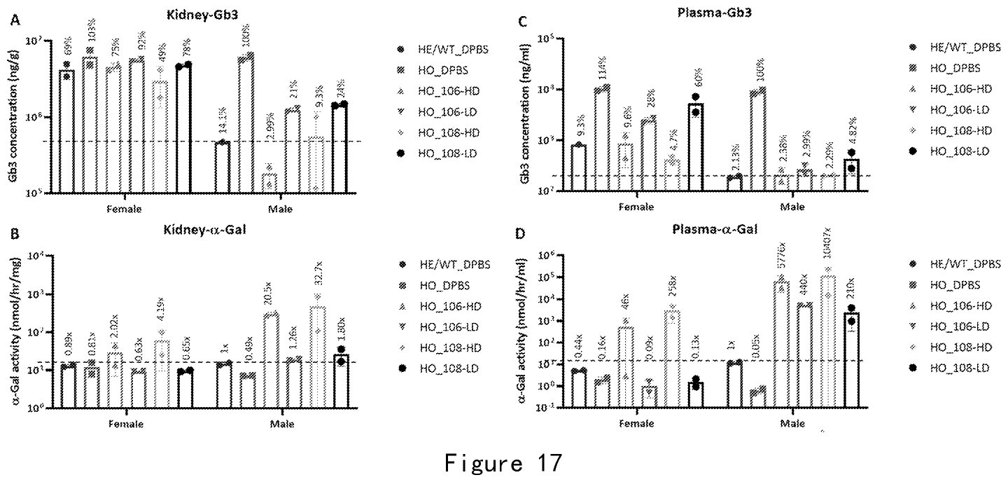

As shown in FIGS. 16 and 17, the treatments of GLA KO Mice with rAAVs 106 and 108 decreased Gb3 accumulation in the heart (FIG. 16C), kidney (FIG. 17A) and plasma (FIG. 17C) in a dose-dependent manner.

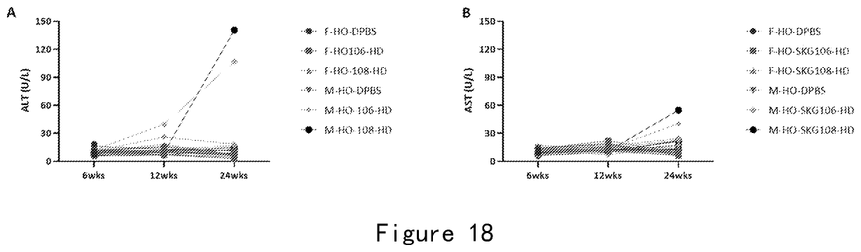

The mice were also tested by ALT (Alanine aminotransferase) and AST (Aspartate aminotransferase) analysis for detecting liver toxicity. The blood ALT and AST levels were measured at 6, 12 and 24 weeks p.i. by the kits from Nanjing Jiancheng (#C009-2-1 for ALT and #C010-2-1 for AST) according to the manufacturer's instructions. The results were shown in FIG. 18. In particular, the ALT and AST levels in the mice injected with the rAAVs were similar to the control mice; although two high-dosed male mice showed abnormal ALT and AST levels at the endpoints (age: 11 months), which was attributed to mice aging, as the other high-dosed male mice did not show abnormal ALT and AST levels. The above results revealed that no liver toxicity after the treatment with rAAV 106 and 108 was observed in GLA KO mice, indicating the safety of the rAAVs.

The tissue samples were further subjected to microscopy. In particular, formalin-fixed tissues were subjected to routine histology processing, including embedded in paraffin, sectioned at 5 μm, and stained with hematoxylin and eosin (H&E). Histopathology evaluation was performed by a board-certified veterinary pathologist at WuXi AppTec. The morphologic results were shown in Table 12.

TABLE 12
The morphologic results of the Pathologist's review.

| Group | Slide ID* | Description of the image | Group | Slide ID | Description of the image |
|---|---|---|---|---|---|
| F_HO_DPBS | G2-1 | No significant abnormality | M_HO_DPBS | G8-1 | No significant abnormality |
| | G2-2 | Lymphocyte infiltration | | G8-2 | No significant abnormality |
| | | was high in the portal area | | | |
| | G2-3 | No significant abnormality | | G8-3 | No significant abnormality |
| | G2-4 | No significant abnormality | | G8-4 | No significant abnormality |
| | G2-5 | No significant abnormality | | G8-5 | Lymphocyte infiltration |
| | | | | | can be observed in part |
| | | | | | of the portal area |
| F_HO_106_HD | G3-1 | Lymphocyte infiltration | M_HO_106_HD | G9-1 | No significant abnormality |
| | | can be observed in the | | | |
| | | portal area | | | |
| | G3-2 | No significant abnormality | | G9-2 | No significant abnormality |
| | G3-3 | No significant abnormality | | G9-3 | No significant abnormality |
| | G3-4 | No significant abnormality | | G9-4 | No significant abnormality |
| | G3-5 | Lymphocyte infiltration | | G9-5 | A number of mature red |
| | | can be observed locally in | | | blood cells are seen |
| | | the portal area | | | locally in the vessels and |
| | | | | | sinusoids |
| | G3-6 | No significant abnormality | | G9-6 | No significant abnormality |
| | G3-7 | No significant abnormality | | G9-7 | No significant abnormality |
| F_HO_108_HD | G5-1 | No significant abnormality | M_HO_108_HD | G11-1 | No significant abnormality |
| | G5-2 | Lymphocyte infiltration | | G11-2 | A number of mature red |
| | | can be observed locally in | | | blood cells are seen |
| | | the portal area | | | locally in the vessels and |
| | | | | | sinusoids |
| | G5-3 | No significant abnormality | | G11-3 | No significant abnormality |
| | G5-4 | No significant abnormality | | G11-4 | No significant abnormality |
| | G5-5 | No significant abnormality | | G11-5 | Lymphocyte infiltration |
| | | | | | was high in the portal |
| | | | | | area |
| | G5-6 | No significant abnormality | | G11-6 | No significant abnormality |
| | G5-7 | No significant abnormality | | G11-7 | A number of fatty |
| | | | | | vacuoles were observed |
| | | | | | locally |
| F_HO_DPBS | G2-1 | No significant abnormality | M_HO_DPBS | G8-1 | No significant abnormality |
| | G2-2 | No significant abnormality | | G8-2 | No significant abnormality |
| | G2-3 | No significant abnormality | | G8-3 | No significant abnormality |
| | G2-4 | No significant abnormality | | G8-4 | Slight inflammatory cell |
| | | | | | infiltration were observed |
| | | | | | locally |
| | G2-5 | No significant abnormality | | G8-5 | Slight steatosis was |
| | | | | | observed in a small |
| | | | | | number of |
| | | | | | cardiomyocytes |
| F_HO_106_HD | G3-1 | No significant abnormality | M_HO_106_HD | G9-1 | No significant abnormality |
| | G3-2 | No significant abnormality | | G9-2 | No significant abnormality |
| | G3-3 | No significant abnormality | | G9-3 | No significant abnormality |
| | G3-4 | A small amount of | | G9-4 | No significant abnormality |
| | | inflammatory cell | | | |
| | | infiltration was observed | | | |
| | | locally | | | |
| | G3-5 | No significant abnormality | | G9-5 | No significant abnormality |
| | G3-6 | No significant abnormality | | G9-6 | No significant abnormality |
| | G3-7 | No significant abnormality | | G9-7 | No significant abnormality |
| F_HO_108_HD | G5-1 | No significant abnormality | M_HO_108_HD | G11-1 | Slight steatosis was |
| | | | | | observed in a small |
| | | | | | number of |
| | | | | | cardiomyocytes |
| | G5-2 | No significant abnormality | | G11-2 | No significant abnormality |
| | G5-3 | No significant abnormality | | G11-3 | No significant abnormality |
| | G5-4 | No significant abnormality | | G11-4 | No significant abnormality |
| | G5-5 | No significant abnormality | | G11-5 | No significant abnormality |
| | G5-6 | Mild hyperplasia and | | G11-6 | No significant abnormality |
| | | slight infiltration of | | | |
| | | inflammatory cell were | | | |
| | | observed in | | | |
| | | cardiomyocytes | | | |
| | G5-7 | No significant abnormality | | G11-7 | No significant abnormality |
| F_HO_DPBS | G2-1 | Infiltration of | M_HO_DPBS | G8-1 | No significant abnormality |
| | | inflammatory cell was | | | |
| | | observed in part of the | | | |
| | | surrounding vessels | | | |
| | G2-2 | Infiltration of | | G8-2 | No significant abnormality |
| | | inflammatory cell was | | | |
| | | observed around vessels | | | |
| | | and part of the renal | | | |
| | | tubules | | | |
| | G2-3 | Mild edema was observed | | G8-3 | Infiltration of |
| | | in a small amount of renal | | | inflammatory cell was |
| | | tubular epithelia | | | observed around part of |
| | | | | | the renal tubules |
| | G2-4 | Infiltration of | | G8-4 | Infiltration of |
| | | inflammatory cell was | | | inflammatory cell was |
| | | observed around part of | | | observed around part of |
| | | the vessels | | | the vessels |
| | G2-5 | Infiltration of | | G8-5 | No significant abnormality |
| | | inflammatory cell was | | | |
| | | observed around part of | | | |
| | | the vessels | | | |
| F_HO_106_HD | G3-1 | Infiltration of | M_HO_106_HD | G9-1 | No significant abnormality |
| | | inflammatory cell was | | | |
| | | observed around the | | | |
| | | vessels and glomerulus | | | |
| | G3-2 | No significant abnormality | | G9-2 | Infiltration of |
| | | | | | inflammatory cell was |
| | | | | | observed around part of |
| | | | | | the vessels |
| | G3-3 | Infiltration of a small | | G9-3 | Infiltration of |
| | | amount of inflammatory | | | inflammatory cell was |
| | | cells was observed around | | | observed around part of |
| | | part of the vessels and | | | the vessels |
| | | glomerulus | | | |
| | G3-4 | No significant abnormality | | G9-4 | Infiltration of |
| | | | | | inflammatory cell was |
| | | | | | observed around part of |
| | | | | | the vessels and |
| | | | | | glomerulus |
| | G3-5 | Infiltration of a small | | G9-5 | Infiltration of |
| | | amount of inflammatory | | | inflammatory cell was |
| | | cells was observed around | | | observed around part of |
| | | part of the vessels and | | | the vessels and |
| | | glomerulus | | | glomerulus |
| | G3-6 | Infiltration of a small | | G9-6 | No significant abnormality |
| | | amount of inflammatory | | | |
| | | cells was observed around | | | |
| | | part of the vessels and | | | |
| | | glomerulus | | | |
| | G3-7 | Infiltration of a small | | G9-7 | Infiltration of |
| | | amount of inflammatory | | | inflammatory cell was |
| | | cells and edema were | | | observed around part of |
| | | observed around part of | | | the vessels and |
| | | renal tube | | | glomerulus |
| F_HO_108_HD | G5-1 | No significant abnormality | M_HO_108_HD | G11-1 | Infiltration of a small |
| | | | | | amount of inflammatory |
| | | | | | cells was observed |
| | | | | | around part of renal tube |
| | G5-2 | No significant abnormality | | G11-2 | Infiltration of |
| | | | | | inflammatory cell was |
| | | | | | observed around part of |
| | | | | | the vessels, and slight |
| | | | | | steatosis was observed in |
| | | | | | part of renal tubular |
| | | | | | epithelia |
| | G5-3 | Infiltration of a great | | G11-3 | No significant abnormality |
| | | number of inflammatory | | | |
| | | cells was observed around | | | |
| | | the vessels and glomerulus | | | |
| | G5-4 | Infiltration of a small | | G11-4 | Infiltration of |
| | | amount of inflammatory | | | inflammatory cell was |
| | | cells was observed around | | | observed around part of |
| | | part of the vessels and | | | renal tube and the vessels |
| | | glomerulus | | | |
| | G5-5 | Slight edema was | | G11-5 | Infiltration of |
| | | observed in part of renal | | | inflammatory cell was |
| | | tubular epithelia | | | observed around part of |
| | | | | | the vessels |
| | G5-6 | No significant abnormality | | G11-6 | Infiltration of |
| | | | | | inflammatory cell was |
| | | | | | observed around part of |
| | | | | | the vessels |
| | G5-7 | Infiltration of a great | | G11-7 | Infiltration of |
| | | number of inflammatory | | | inflammatory cell was |
| | | cells was observed around | | | observed around part of |
| | | part of renal tube | | | the vessels |

*Indicating the animal, from which the sample was derived, e.g., Slide ID G2-n referred to samples derived from G2.

It can be seen that the treatment of rAAVs 106 and 108 did not result in significant abnormality in morphology of the tissue samples as compared to the controls.

### Example 5. Treatment with rAAV 108 in Fabry Aggravated Model

TgG3S mice (transgenic mice expressing human Gb3 Synthase, purchased from JCRB Laboratory Animal Resource Bank, National Institutes of Biomedical Innovation, Health and Nutrition in Japan) were crossed with GLA KO mice to obtain Fabry aggravated models (TgG3S×GLA KO).

The rAAV 108 was diluted in formulation buffer and injected into the tail veins of 8-week-old Fabry aggravated models at high dose (HD) of 1.25E13 vg/kg or low dose (LD) of 1.25E12 vg/kg. The overexpression of TgG3S increased the synthesis of Gb3 in vivo. The treatments of the mice were shown in Table 13.

TABLE 13

| | | | | | Dose | Dose (vg/ | Number of | i.v. injection |
|---|---|---|---|---|---|---|---|---|
| Group | Gender | TgG3S | GLA | Treatment | (vg/kg) | 20 g mouse) | animals | (μl/mouse) |
| G1 | Male | + | WT | DPBS + 0.001% | 0 | 0 | 7 | 200 |
| | | | | PF68 | | | | |
| G2 | Male | + | HO | DPBS + 0.001% | 0 | 0 | 7 | 200 |
| | | | | PF68 | | | | |
| G3 | Male | + | HO | rAAV 108 | 1.25E+13 | 2.50E+11 | 7 | Depends on BW |
| G4 | Male | + | HO | rAAV 108 | 1.25E+12 | 2.50E+10 | 7 | Depends on BW |

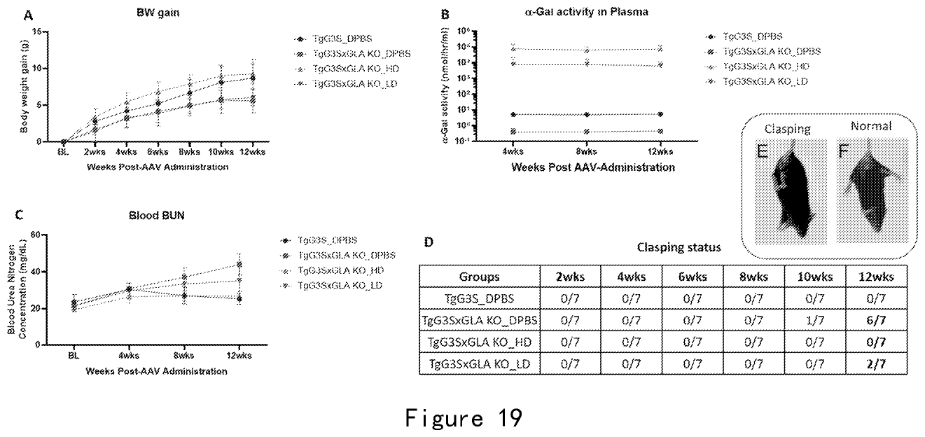

The measurement of body weight gain (as described in Example 4) and the detection of clasping (the mouse tucks its hind limbs to the body when suspended by the tail, FIGS. 19E and 19F, indicating the accumulation of Gb3) status were carried out bi-weekly till 12 weeks p.i., and the plasma (4, 8 and 12 weeks post injection) and urine (5, 9 and 13 weeks post injection) were collected. The GLA activity in plasma, blood urea nitrogen (BUN), urine albumin, urine creatinine and urine osmolality were measured. The BUN was measured with Thermo #EIABUN kit; the urine albumin was measured with Abcam #ab207327 kit; the urine creatinine was measured with Abcam #ab204537 kit; and the urine osmolality was determined with OsmoTECH® Single-Sample Micro-Osmometer (Advanced Instruments). The measurements and determination were carried out according to the manufacturers' instructions.

The results showed that the body weight gain did not significantly vary between groups (FIG. 19A). The GLA activities in plasma were stably increased by the treatment of rAAV 108 in a dose-dependent manner (FIG. 19B). The BUN was decreased by the treatment of rAAV 108 in a dose-dependent manner, and the HD group achieved a BUN level similar to the group that was WT for GLA (FIG. 19C).

6 of 7 non-treated TgG3S×GLA KO mice showed clasping at 12 weeks p.i., while all of the mice that were WT for GLA, and that treated with HD rAAV 108 were normal, and 2 of 7 mice in LD group showed clasping at 12 weeks p.i. (FIG. 19D), indicating the effect of rAAV 108 in a dose-dependent manner.

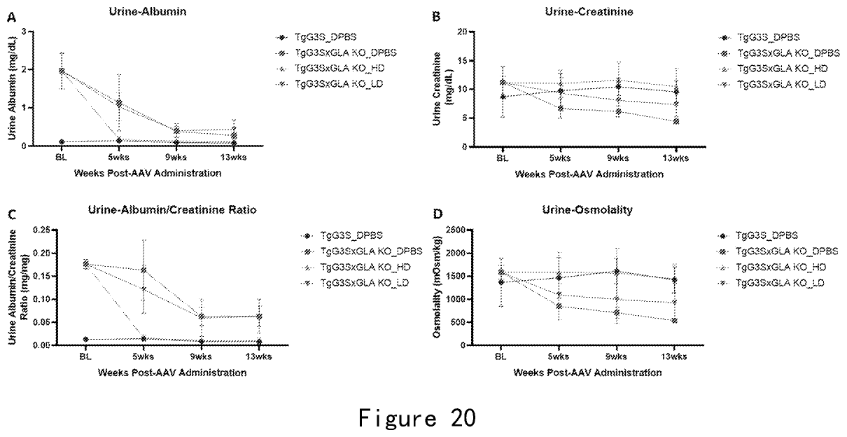

As shown in FIG. 20, the urine albumin was decreased by the treatment of rAAV 108 in a dose-dependent manner, and the HD group achieved a similar urine albumin level since 5 weeks p.i. (FIG. 20A). The urine creatinine, the ratio of albumin to creatinine, and the urine osmolality showed a similar pattern, i.e., the treatment of rAAV 108 contributed to the recovery of renal function in a dose-dependent manner (FIGS. 20B, 20C and 20D).

The mice were grown till 40 weeks p.i, and the blood and urine were collected every 4 weeks, and analyzed as described above.

At the end of the study, the tissues (kidney, liver, heart, spinal cord, dorsal root ganglia (DRG), lung, spleen quadriceps, etc.) were collected and analyzed as described previously.

### REFERENCES

1. Zarate Y A, Hopkin R J. Fabry's disease. Lancet. 2008; 372:1427-35.
2. Lenders M, Brand E. Fabry Disease: The Current Treatment Landscape. Drugs. 2021; 81:635-645.
3. Eng C M, Fletcher J, Wilcox W R, Waldek S, Scott C R, Sillence D O, et al. Fabry disease: baseline medical characteristics of a cohort of 1765 males and females in the Fabry Registry. J Inherit Metab Dis. 2007; 30:184-92.
4. Echevarria L, Benistan K, Toussaint A, Dubourg O, Hagege A A, Eladari D, et al.

X-chromosome inactivation in female patients with Fabry disease. Clin Genet. 2016; 89:44-54.
5. Waldek S, Patel M R, Banikazemi M, Lemay R, Lee P. Life expectancy and cause of death in males and females with Fabry disease: Findings from the Fabry Registry. Genetics in Medicine. 2009; 11:790-796.
6. Feriozzi S, Hughes D A. New drugs for the treatment of Anderson-Fabry disease. Journal of Nephrology. 2021; 34:221-230.
7. Eng C M, Guffon N, Wilcox W R, Germain D P, Lee P, Waldek S, et al. Safety and efficacy of recombinant human a-galactosidase A replacement therapy in Fabry's disease. N Engl J Med. 2001; 345:9-16.
8. Schiffmann R, Kopp J B, Austin H A 3rd, Sabnis S, Moore D F, Weibel T, et al. Enzyme replacement therapy in Fabry disease: a randomized controlled trial. JAMA. 2001; 285:2743-9.
9. Rombach S M, Smid B E, Bouwman M G, Linthorst G E, et al. Long term enzyme replacement therapy for Fabry disease: effectiveness on kidney, heart and brain. Orphanet Journal of Rare Diseases, 2013; 8:47.
10. McCafferty E H, Scott U. Migalastat: A review in Fabry disease. Drugs. 2019; 79:543-54.
11. Germain D P, Hughes D A, Nicholls K, Bichet D G, Giugliani R, Wilcox W R, et al. Treatment of Fabry's disease with the pharmacologic chaperone migalastat. N Engl J Med. 2016; 375:545-55.
12. Crosson S M, Dib P, Smith J K, Zolotukhin S. Helper-free Production of Laboratory Grade AAV and Purification by Iodixanol Density Gradient Centrifugation. Mol Ther Methods Clin Dev. 2018; 10:1-7.

Sequence Listing

SEQ ID NO: 1 hGLA.co1
ATGCAGCTGAGAAATCCTGAGCTGCACCTGGGCTGTGCCCTGGCTCTGAGATTTCTGGCTCTGGTGTCCTG
GGACATCCCTGGCGCTAGAGCCCTGGATAACGGCCTGGCCAGAACACCTACAATGGGCTGGCTGCACTGGG
AGAGATTCATGTGCAACCTGGACTGCCAAGAGGAACCCGACAGCTGCATCAGCGAGAAGCTGTTCATGGAA
ATGGCCGAGCTGATGGTGTCCGAAGGCTGGAAGGACGCCGGCTACGAGTACCTGTGCATCGACGACTGTTG
GATGGCCCCTCAGAGAGACTCTGAGGGCAGACTGCAGGCCGATCCTCAGAGATTTCCCCACGGCATTAGAC
AGCTGGCCAACTACGTGCACAGCAAGGGCCTGAAGCTGGGCATCTACGCCGACGTGGGCAACAAGACCTGT
GCCGGCTTTCCTGGCAGCTTCGGCTACTACGATATCGACGCCCAGACCTTCGCCGATTGGGGAGTCGATCT
GCTGAAGTTCGACGGCTGCTACTGCGACAGCCTGGAAAATCTGGCCGACGGCTACAAGCACATGTCTCTGG
CCCTGAATCGGACCGGCAGATCCATCGTGTACAGCTGCGAGTGGCCCCTGTACATGTGGCCCTTCCAGAAG
CCTAACTACACCGAGATCAGACAGTACTGCAACCACTGGCGGAACTTCGCCGACATCGACGATAGCTGGAA
GTCCATCAAGAGCATCCTGGACTGGACCAGCTTCAATCAAGAGCGGATCGTGGACGTGGCAGGACCTGGCG
GATGGAACGATCCTGACATGCTGGTCATCGGCAACTTCGGCCTGAGCTGGAACCAGCAAGTGACCCAGATG
GCCCTGTGGGCCATTATGGCCGCTCCTCTGTTCATGAGCAACGACCTGAGACACATCAGCCCTCAGGCCAA
GGCTCTGCTGCAGGACAAGGATGTGATCGCTATCAACCAGGATCCTCTGGGCAAGCAGGGCTACCAGCTGA
GACAGGGCGACAATTTCGAAGTGTGGGAAAGACCCCTGAGCGGACTGGCTTGGGCCGTCGCCATGATCAAC
AGACAAGAGATCGGCGGACCCCGGTCCTACACAATTGCCGTGGCTTCTCTCGGCAAAGGCGTGGCCTGTAA
TCCCGCCTGCTTTATCACACAGCTGCTGCCCGTGAAGAGAAAGCTGGGCTTTTACGAGTGGACCAGCAGAC
TGCGGAGCCACATCAATCCTACCGGCACAGTGCTGCTGCAGCTGGAAAACACAATGCAGATGAGCCTGAAG
GACCTGCTGTGATGA
SEQ ID NO: 2 hGLA.co2
ATGCAGCTCCGAAACCCGGAATTGCATTTGGGCTGTGCGCTGGCGCTCAGATTTCTGGCACTCGTCTCTTG
GGACATTCCTGGGGCGCGGGCGCTCGACAACGGCCTGGCACGAACCCCAACGATGGGCTGGTTGCATTGGG
AGCGGTTTATGTGTAACCTTGACTGTCAAGAGGAACCGGATAGCTGTATCAGTGAGAAGCTGTTTATGGAA
ATGGCAGAATTGATGGTGTCCGAAGGGTGGAAAGACGCGGGATACGAGTACCTCTGCATCGACGACTGCTG
GATGGCACCCCAGCGGGACTCAGAAGGGAGGCTGCAGGCTGACCCTCAACGGTTTCCCCACGGGATAAGAC
AACTGGCGAACTACGTTCATTCCAAAGGACTCAAACTGGGTATCTACGCTGACGTCGGAAATAAGACGTGT
GCGGGCTTTCCAGGGAGCTTCGGGTACTACGACATCGACGCCCAAACGTTTGCGGACTGGGGCGTTGACTT
GCTCAAATTTGACGGCTGCTACTGTGACAGCCTGGAAAATCTGGCAGATGGCTATAAACATATGAGTCTCG
CCCTTAACCGGACTGGTAGATCAATTGTATATTCCTGCGAGTGGCCTTTGTACATGTGGCCATTCCAGAAA
CCGAACTATACTGAGATACGCCAATATTGCAATCATTGGCGAAATTTCGCCGACATAGATGATAGTTGGAA
GTCTATAAAGTCTATTCTTGATTGGACTTCTTTTAACCAGGAGCGCATTGTGGACGTTGCGGGGCCCGGAG
GGTGGAACGATCCCGATATGCTTGTGATCGGCAACTTCGGTCTGTCCTGGAACCAACAAGTCACCCAAATG
GCATTGTGGGCGATAATGGCTGCGCCCCTGTTCATGTCTAATGATCTCAGGCACATAAGCCCCCAGGCTAA
AGCGCTTCTTCAAGATAAGGACGTTATAGCTATCAACCAGGACCCCCTTGGGAAGCAGGGCTACCAGCTTC
GACAAGGAGATAATTTCGAGGTGTGGGAGCGCCCCCTGTCCGGGCTTGCGTGGGCGGTCGCCATGATCAAC
CGACAGGAAATTGGGGGCCCCAGATCATATACGATCGCAGTTGCTAGCCTCGGAAAAGGCGTTGCTTGCAA
TCCCGCTTGCTTCATTACGCAACTCCTCCCCGTTAAGCGGAAACTGGGATTCTACGAGTGGACATCCCGGC
TGCGATCACACATAAATCCCACTGGGACAGTCCTCCTCCAGCTGGAGAATACAATGCAAATGAGCCTTAAA
GACCTCTTGTGATGA
SEQ ID NO: 3 hGLA.co3
ATGCAGCTGAGGAACCCAGAGCTGCACCTGGGCTGCGCACTGGCCCTGAGATTCCTGGCCCTGGTGTCCTG
GGACATCCCTGGAGCACGGGCCCTGGATAACGGACTGGCAAGAACCCCAACAATGGGCTGGCTGCACTGGG
AGCGGTTCATGTGCAATCTGGACTGTCAGGAGGAGCCAGATAGCTGTATCTCCGAGAAGCTGTTTATGGAG
ATGGCCGAGCTGATGGTGTCTGAGGGCTGGAAGGACGCCGGCTACGAGTATCTGTGCATCGACGATTGTTG
GATGGCACCACAGAGGGACAGCGAGGGCAGACTGCAGGCCGATCCCCAGAGGTTTCCTCACGGCATCCGCC
AGCTGGCCAACTACGTGCACAGCAAGGGCCTGAAGCTGGGCATCTACGCCGACGTGGGCAATAAGACCTGC
GCCGGCTTCCCTGGCTCCTTTGGCTACTACGACATCGACGCCCAGACATTCGCCGACTGGGGCGTGGATCT
GCTGAAGTTTGACGGCTGCTACTGTGATTCCCTGGAGAACCTGGCCGACGGCTATAAGCACATGTCTCTGG
CCCTGAATAGGACCGGCCGCTCTATCGTGTACAGCTGCGAGTGGCCACTGTATATGTGGCCATTCCAGAAG
CCCAACTACACAGAGATCCGGCAGTATTGTAACCACTGGAGAAATTTTGCCGACATCGACGATTCTTGGAA
GAGCATCAAGTCCATCCTGGACTGGACCTCCTTCAACCAGGAGAGGATCGTGGACGTGGCAGGACCTGGAG
GCTGGAACGACCCAGATATGCTGGTCATCGGCAACTTCGGCCTGTCTTGGAATCAGCAGGTGACACAGATG
GCACTGTGGGCAATCATGGCAGCACCACTGTTTATGAGCAATGATCTGAGGCACATCTCCCCACAGGCAAA
GGCCCTGCTGCAGGACAAGGATGTGATCGCCATCAACCAGGACCCCCTGGGCAAGCAGGGCTACCAGCTGA
GGCAGGGCGATAATTTTGAAGTGTGGGAGAGGCCACTGTCCGGACTGGCCTGGGCCGTGGCCATGATCAAC
AGGCAGGAGATCGGCGGCCCTCGCTCTTACACCATCGCAGTGGCAAGCCTGGGCAAGGGAGTGGCATGCAA
TCCAGCATGTTTCATCACCCAGCTGCTGCCTGTGAAGCGGAAGCTGGGCTTTTATGAGTGGACATCCCGGC
TGAGATCTCACATCAACCCTACCGGCACAGTGCTGCTGCAGCTGGAGAATACAATGCAGATGAGCCTGAAG
GACCTGCTGTGATGA
SEQ ID NO: 4 hGLA.co4
ATGCAGCTGAGAAACCCCGAGCTGCACCTGGGCTGCGCCCTGGCCCTGAGATTCCTGGCCCTGGTGAGCTG
GGACATCCCCGGCGCCAGAGCCCTGGACAACGGCCTGGCCAGAACCCCCACCATGGGCTGGCTGCACTGGG
AGAGATTCATGTGCAACCTGGACTGCCAGGAGGAGCCCGACAGCTGCATCAGCGAGAAGCTGTTCATGGAG
ATGGCCGAGCTGATGGTGAGCGAGGGCTGGAAGGACGCCGGCTACGAGTACCTGTGCATCGACGACTGCTG
GATGGCCCCCCAGAGAGACAGCGAGGGCAGACTGCAGGCCGACCCCCAGAGATTCCCCCACGGCATCAGAC
AGCTGGCCAACTACGTGCACAGCAAGGGCCTGAAGCTGGGCATCTACGCCGACGTGGGCAACAAGACCTGC
GCCGGCTTCCCCGGCAGCTTCGGCTACTACGACATCGACGCCCAGACCTTCGCCGACTGGGGCGTGGACCT
GCTGAAGTTCGACGGCTGCTACTGCGACAGCCTGGAGAACCTGGCCGACGGCTACAAGCACATGAGCCTGG
CCCTGAACAGAACCGGCAGAAGCATCGTGTACAGCTGCGAGTGGCCCCTGTACATGTGGCCCTTCCAGAAG
CCGAACTACACCGAGATCAGACAGTACTGCAACCACTGGAGAAACTTCGCCGACATCGACGACAGCTGGAA
GAGCATCAAGAGCATCCTGGACTGGACCAGCTTCAACCAGGAGAGAATCGTGGACGTGGCCGGCCCCGGCG
GCTGGAACGACCCCGACATGCTGGTGATCGGCAACTTCGGCCTGAGCTGGAACCAGCAGGTGACCCAGATG
GCCCTGTGGGCCATCATGGCCGCCCCCCTGTTCATGAGCAACGACCTGAGACACATCAGCCCCCAGGCCAA
GGCCCTGCTGCAGGACAAGGACGTGATCGCCATCAACCAGGACCCCCTGGGCAAGCAGGGCTACCAGCTGA
GACAGGGCGACAACTTCGAGGTGTGGGAGAGACCCCTGAGCGGCCTGGCCTGGGCCGTGGCCATGATCAAC
AGACAGGAGATCGGCGGCCCCAGAAGCTACACCATCGCCGTGGCCAGCCTGGGCAAGGGCGTGGCCTGCAA
CCCCGCCTGCTTCATCACCCAGCTGCTGCCCGTGAAGAGAAAGCTGGGCTTCTACGAGTGGACCAGCAGAC
TGAGAAGTCACATCAACCCCACCGGCACCGTGCTGCTGCAGCTGGAGAACACCATGCAGATGAGCCTGAAG
GACCTGCTGTGATGA
SEQ ID NO: 5 Chicken beta-actin (CBA) chimeric intron (intron1)
GAGTCGCTGCGCGCTGCCTTCGCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCCGGGCTGTAATTAGCGCTTGGT
TTAATGACGGCTTGTTTCTTTTCTGTGGCTGCGTGAAAGCCTTGAGGGGCTCCGGGAGGGCCCTTTGTGCG
GGGGGAGCGGCTCGGGGGGTGCGTGCGTGTGTGTGTGCGTGGGGAGCGCCGCGTGCGGCTCCGCGCTGCCC
GGCGGCTGTGAGCGCTGCGGGCGCGGCGCGGGGCTTTGTGCGCTCCGCAGTGTGCGCGAGGGGAGCGCGGC
CGGGGGCGGTGCCCCGCGGTGCGGGGGGGGCTGCGAGGGGAACAAAGGCTGCGTGCGGGGTGTGTGCGTGG
GGGGGTGAGCAGGGGGTGTGGGCGCGTCGGTCGGGCTGCAACCCCCCCTGCACCCCCCTCCCCGAGTTGCT
GAGCACGGCCCGGCTTCGGGTGCGGGGCTCCGTACGGGGCGTGGCGCGGGGCTCGCCGTGCCGGGCGGGGG
GTGGCGGCAGGTGGGGGTGCCGGGGGGGGGGGGGCCGCCTCGGGCCGGGGAGGGCTCGGGGGAGGGGCGCG
GCGGCCCCCGGAGCGCCGGCGGCTGTCGAGGCGCGGCGAGCCGCAGCCATTGCCTTTTATGGTAATCGTGC
GAGAGGGCGCAGGGACTTCCTTTGTCCCAAATCTGTGCGGAGCCGAAATCTGGGAGGCGCCGCCGCACCCC
CTCTAGCGGGCGCGGGGCGAAGCGGTGCGGCGCCGGCAGGAAGGAAATGGGGGGGGAGGGCCTTCGTGCGT
CGCCGCGCCGCCGTCCCCTTCTCCCTCTCCAGCCTCGGGGCTGTCCGCGGGGGGACGGCTGCCTTCGGGGG
GGACGGGGCAGGGCGGGGTTCGGCTTCTGGCGTGTGACCGGCGGCTCTAGAGCCTCTGCTAACCATGTTCA
TGCCTTCTTCTTTTTCCTACAGCTCCTGGGCAACGTGCTGGTTATTGTGCTGTCTCATCATTTTGGCAAAG
SEQ ID NO: 6 Modified CBA chimeric intron (intron2)
GAGTCGCTGCGCGCTGCCTTCGCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCCGGGCTGTAATTAGCGCTTGGT
TTAATGACGGCTTGTTTCTTTTCTGTGGCTGCGTGAAAGCCTTGAGGGGCTCCGGGAGGGCCCTTTGTGCG
GGGGGAGCGGCTCGGGGCTGTCCGCGGGGGGACGGCTGCCTTCGGGGGGGACGGGGCAGGGCGGGGTTCGG
CTTCTGGCGTGTGACCGGCGGCTCTAGAGCCTCTGCTAACCATGTTCATGCCTTCTTCTTTTTCCTACAGC
TCCTGGGCAACGTGCTGGTTATTGTGCTGTCTCATCATTTTGGCAAAG
SEQ ID NO: 7 Promega chimeric intron (intron3)
GTAAGTATCAAGGTTACAAGACAGGTTTAAGGAGACCAATAGAAACTGGGCTTGTCGAGACAGAGAAGACT
CTTGCGTTTCTGATAGGCACCTATTGGTCTTACTGACATCCACTTTGCCTTTCTCTCCACAG
SEQ ID NO: 8 SV40 intron (intron4)
AACTGAAAAACCAGAAAGTTAACTGGTAAGTTTAGTCTTTTTGTCTTTTATTTCAGGTCCCGGATCCGGTG
GTGGTGCAAATCAAAGAACTGCTCCTCAGTGGATGTTGCCTTTACTTCTAGGCCTGTACGGAAGTGTTACT
TCTGCTCTAAAAGCT
SEQ ID NO: 9 CMV early enhancer
CTAGTCGACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACGTCAATGGGTGGAGT
ATTTACGGTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTTGGCAGTA
CATCTACGTATTAGTCATCGCTATTAC
SEQ ID NO: 10 chicken beta-actin core promoter
TGGTCGAGGTGAGCCCCACGTTCTGCTTCACTCTCCCCATCTCCCCCCCCTCCCCACCCCCAATTTTGTAT
TTATTTATTTTTTAATTATTTTGTGCAGCGATGGGGGCGGGGGGGGGGGGGGGGCGCGCGCCAGGCGGGGC
GGGGCGGGGCGAGGGGCGGGGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGAAGCGCGCGGCGGGCGG
SEQ ID NO: 11 RBG polyA signal sequence
GATCTTTTTCCCTCTGCCAAAAATTATGGGGACATCATGAAGCCCCTTGAGCATCTGACTTCTGGCTAATA
AAGGAAATTTATTTTCATTGCAATAGTGTGTTGGAATTTTTTGTGTCTCTCACTCG
SEQ ID NO: 12 human α-Gal A
MQLRNPELHLGCALALRFLALVSWDIPGARALDNGLARTPTMGWLHWEREMCNLDCQEEPDSCISEKLFMEMAEL
MVSEGWKDAGYEYLCIDDCWMAPQRDSEGRLQADPQRFPHGIRQLANYVHSKGLKLGIYADVGNKTCAGFPGSFG
YYDIDAQTFADWGVDLLKFDGCYCDSLENLADGYKHMSLALNRTGRSIVYSCEWPLYMWPFQKPNYTEIRQYCNH
WRNFADIDDSWKSIKSILDWTSFNQERIVDVAGPGGWNDPDMLVIGNFGLSWNQQVTQMALWAIMAAPLFMSNDL
RHISPQAKALLQDKDVIAINQDPLGKQGYQLRQGDNFEVWERPLSGLAWAVAMINRQEIGGPRSYTIAVASLGKG
VACNPACFITQLLPVKRKLGFYEWTSRLRSHINPTGTVLLQLENTMQMSLKDLL
SEQ ID NO: 13 pGCB 108
CCTGCAGGCAGCTGCGCGCTCGCTCGCTCACTGAGGCCGCCCGGGCAAAGCCCGGGCGTCGGGCGACCTTT
GGTCGCCCGGCCTCAGTGAGCGAGCGAGCGCGCAGAGAGGGAGTGGCCAACTCCATCACTAGGGGTTCCTG
CGGCCGCACGCGTCAATTGCTAGTCGACATTGATTATTGACTAGTTATTAATAGTAATCAATTACGGGGTC
ATTAGTTCATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGCCAAG
TACGCCCCCTATTGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTCGAGGTGAGCCCCACGTTC
TGCTTCACTCTCCCCATCTCCCCCCCCTCCCCACCCCCAATTTTGTATTTATTTATTTTTTAATTATTTTG
TGCAGCGATGGGGGCGGGGGGGGGGGGGGGGGGGCGCGCCAGGCGGGGGGGGGGGGGCGAGGGGCGGGGCG
GGGCGAGGCGGAGAGGTGCGGCGGCAGCCAATCAGAGCGGCGCGCTCCGAAAGTTTCCTTTTATGGCGAGG
CGGCGGCGGCGGCGGCCCTATAAAAAGCGAAGCGCGCGGCGGGCGGAAGCTTGAGTCGCTGCGCGCTGCCT
TCGCCCCGTGCCCCGCTCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTAATGACGGCTTGTTTCT
TTTCTGTGGCTGCGTGAAAGCCTTGAGGGGCTCCGGGAGGGCCCTTTGTGCGGGGGGAGCGGCTCGGGGCT
GTCCGCGGGGGGACGGCTGCCTTCGGGGGGGACGGGGCAGGGCGGGGTTCGGCTTCTGGCGTGTGACCGGC
GGCTCTAGAGCCTCTGCTAACCATGTTCATGCCTTCTTCTTTTTCCTACAGCTCCTGGGCAACGTGCTGGT
TATTGTGCTGTCTCATCATTTTGGCAAAGAATTGGCCTCGAGGACGGGGTGAACTACGCCTGAGGATCCGC
CACCATGCAGCTGAGAAACCCCGAGCTGCACCTGGGCTGCGCCCTGGCCCTGAGATTCCTGGCCCTGGTGA
GCTGGGACATCCCCGGCGCCAGAGCCCTGGACAACGGCCTGGCCAGAACCCCCACCATGGGCTGGCTGCAC
TGGGAGAGATTCATGTGCAACCTGGACTGCCAGGAGGAGCCCGACAGCTGCATCAGCGAGAAGCTGTTCAT
GGAGATGGCCGAGCTGATGGTGAGCGAGGGCTGGAAGGACGCCGGCTACGAGTACCTGTGCATCGACGACT
GCTGGATGGCCCCCCAGAGAGACAGCGAGGGCAGACTGCAGGCCGACCCCCAGAGATTCCCCCACGGCATC
AGACAGCTGGCCAACTACGTGCACAGCAAGGGCCTGAAGCTGGGCATCTACGCCGACGTGGGCAACAAGAC
CTGCGCCGGCTTCCCCGGCAGCTTCGGCTACTACGACATCGACGCCCAGACCTTCGCCGACTGGGGCGTGG
ACCTGCTGAAGTTCGACGGCTGCTACTGCGACAGCCTGGAGAACCTGGCCGACGGCTACAAGCACATGAGC
CTGGCCCTGAACAGAACCGGCAGAAGCATCGTGTACAGCTGCGAGTGGCCCCTGTACATGTGGCCCTTCCA
GAAGCCGAACTACACCGAGATCAGACAGTACTGCAACCACTGGAGAAACTTCGCCGACATCGACGACAGCT
GGAAGAGCATCAAGAGCATCCTGGACTGGACCAGCTTCAACCAGGAGAGAATCGTGGACGTGGCCGGCCCC
GGCGGCTGGAACGACCCCGACATGCTGGTGATCGGCAACTTCGGCCTGAGCTGGAACCAGCAGGTGACCCA
GATGGCCCTGTGGGCCATCATGGCCGCCCCCCTGTTCATGAGCAACGACCTGAGACACATCAGCCCCCAGG
CCAAGGCCCTGCTGCAGGACAAGGACGTGATCGCCATCAACCAGGACCCCCTGGGCAAGCAGGGCTACCAG
CTGAGACAGGGCGACAACTTCGAGGTGTGGGAGAGACCCCTGAGCGGCCTGGCCTGGGCCGTGGCCATGAT
CAACAGACAGGAGATCGGCGGCCCCAGAAGCTACACCATCGCCGTGGCCAGCCTGGGCAAGGGCGTGGCCT
GCAACCCCGCCTGCTTCATCACCCAGCTGCTGCCCGTGAAGAGAAAGCTGGGCTTCTACGAGTGGACCAGC
AGACTGAGAAGTCACATCAACCCCACCGGCACCGTGCTGCTGCAGCTGGAGAACACCATGCAGATGAGCCT
GAAGGACCTGCTGTGATGATGTACAGGTACCTCGATGGATTTCCAAGGTTAATTCATTGGAATTGAAAATT
AACAGAGATCTAGAGCTGAATTCCTGCAGCCAGGGGGATCAGCCTGATCTTTTTCCCTCTGCCAAAAATTA
TGGGGACATCATGAAGCCCCTTGAGCATCTGACTTCTGGCTAATAAAGGAAATTTATTTTCATTGCAATAG
TGTGTTGGAATTTTTTGTGTCTCTCACTCGCGGACCGAGCGGCCGCAGGAACCCCTAGTGATGGAGTTGGC
CACTCCCTCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAGCTGCCTGCAGGGGCAGCTTGAAGGAAATACTAAGGCA
AAGGTACTGCAAGTGCTCGCAACATTCGCTTATGCGGATTATTGCCGTAGTGCCGCGACGCCGGGGGCAAG
ATGCAGAGATTGCCATGGTACAGGCCGTGCGGTTGATATTGCCAAAACAGAGCTGTGGGGGAGAGTTGTCG
AGAAAGAGTGCGGAAGATGCAAAGGCGTCGGCTATTCAAGGATGCCAGCAAGCGCAGCATATCGCGCTGTG
ACGATGCTAATCCCAAACCTTACCCAACCCACCTGGTCACGCACTGTTAAGCCGCTGTATGACGCTCTGGT
GGTGCAATGCCACAAAGAAGAGTCAATCGCAGACAACATTTTGAATGCGGTCACACGTTAGCAGCATGATT
GCCACGGATGGCAACATATTAACGGCATGATATTGACTTATTGAATAAAATTGGGTAAATTTGACTCAACG
ATGGGTTAATTCGCTCGTTGTGGTAGTGAGATGAAAAGAGGCGGCGCTTACTACCGATTCCGCCTAGTTGG
TCACTTCGACGTATCGTCTGGAACTCCAACCATCGCAGGCAGAGAGGTCTGCAAAATGCAATCCCGAAACA
GTTCGCAGGTAATAGTTAGAGCCTGCATAACGGTTTCGGGATTTTTTATATCTGCACAACAGGTAAGAGCA
TTGAGTCGATAATCGTGAAGAGTCGGCGAGCCTGGTTAGCCAGTGCTCTTTCCGTTGTGCTGAATTAAGCG
AATACCGGAAGCAGAACCGGATCACCAAATGCGTACAGGCGTCATCGCCGCCCAGCAACAGCACAACCCAA
ACTGAGCCGTAGCCACTGTCTGTCCTGAATTCATTAGTAATAGTTACGCTGCGGCCTTTTACACATGACCT
TCGTGAAAGCGGGTGGCAGGAGGTCGCGCTAACAACCTCCTGCCGTTTTGCCCGTGCATATCGGTCACGAA
CAAATCTGATTACTAAACACAGTAGCCTGGATTTGTTCTATCAGTAATCGACCTTATTCCTAATTAAATAG
AGCAAATCCCCTTATTGGGGGTAAGACATGAAGATGCCAGAAAAACATGACCTGTTGGCCGCCATTCTCGC
GGCAAAGGAACAAGGCATCGGGGCAATCCTTGCGTTTGCAATGGCGTACCTTCGCGGCAGATATAATGGCG
GTGCGTTTACAAAAACAGTAATCGACGCAACGATGTGCGCCATTATCGCCTGGTTCATTCGTGACCTTCTC
GACTTCGCCGGACTAAGTAGCAATCTCGCTTATATAACGAGCGTGTTTATCGGCTACATCGGTACTGACTC
GATTGGTTCGCTTATCAAACGCTTCGCTGCTAAAAAAGCCGGAGTAGAAGATGGTAGAAATCAATAATCAA
CGTAAGGCGTTCCTCGATATGCTGGCGTGGTCGGAGGGAACTGATAACGGACGTCAGAAAACCAGAAATCA
TGGTTATGACGTCATTGTAGGCGGAGAGCTATTTACTGATTACTCCGATCACCCTCGCAAACTTGTCACGC
TAAACCCAAAACTCAAATCAACAGGCGCCGGACGCTACCAGCTTCTTTCCCGTTGGTGGGATGCCTACCGC
AAGCAGCTTGGCCTGAAAGACTTCTCTCCGAAAAGTCAGGACGCTGTGGCATTGCAGCAGATTAAGGAGCG
TGGCGCTTTACCTATGATTGATCGTGGTGATATCCGTCAGGCAATCGACCGTTGCAGCAATATCTGGGCTT
CACTGCCGGGCGCTGGTTATGGTCAGTTCGAGCATAAGGCTGACAGCCTGATTGCAAAATTCAAAGAAGCG
GGCGGAACGGTCAGAGAGATTGATGTATGAGCAGAGTCACCGCGATTATCTCCGCTCTGGTTATCTGCATC
ATCGTCTGCCTGTCATGGGCTGTTAATCATTACCGTGATAACGCCATTACCTACAAAGCCCAGCGCGACAA
AAATGCCAGAGAACTGAAGCTGGCGAACGCGGCAATTACTGACATGCAGATGCGTCAGCGTGATGTTGCTG
CGCTCGATGCAAAATACACGAAGGAGTTAGCTGATGCTAAAGCTGAAAATGATGCTCTGCGTGATGATGTT
GCCGCTGGTCGTCGTCGGTTGCACATCAAAGCAGTCTGTCAGTCAGTGCGTGAAGCCACCACCGCCTCCGG
CGTGGATAATGCAGCCTCCCCCCGACTGGCAGACACCGCTGAACGGGATTATTTCACCCTCAGAGAGAGGC
TGATCACTATGCAAAAACAACTGGAAGGAACCCAGAAGTATATTAATGAGCAGTGCAGATAGAGTTGCCCA
TATCGATGGGCAACTCATGCAATTATTGTGAGCAATACACACGCGCTTCCAGCGGAGTATAAATGCCTAAA
GTAATAAAACCGAGCAATCCATTTACGAATGTTTGCTGGGTTTCTGTTTTAACAACATTTTCTGCGCCGCC
ACAAATTTTGGCTGCATCGACAGTTTTCTTCTGCCCAATTCCAGAAACGAAGAAATGATGGGTGATGGTTT
CCTTTGGTGCTACTGCTGCCGGTTTGTTTTGAACAGTAAACGTCTGTTGAGCACATCCTGTAATAAGCAGG
GCCAGCGCAGTAGCGAGTAGCATTTTTTTCATGGTGTTATTCCCGATGCTTTTTGAAGTTCGCAGAATCGT
ATGTGTAGAAAATTAAACAAACCCTAAACAATGAGTTGAAATTTCATATTGTTAATATTTATTAATGTATG
TCAGGTGCGATGAATCGTCATTGTATTCCCGGATTAACTATGTCCACAGCCCTGACGGGGAACTTCTCTGC
GGGAGTGTCCGGGAATAATTAAAACGATGCACACAGGGTTTAGCGCGTACACGTATTGCATTATGCCAACG
CCCCGGTGCTGACACGGAAGAAACCGGACGTTATGATTTAGCGTGGAAAGATTTGTGTAGTGTTCTGAATG
CTCTCAGTAAATAGTAATGAATTATCAAAGGTATAGTAATATCTTTTATGTTCATGGATATTTGTAACCCA
TCGGAAAACTCCTGCTTTAGCAAGATTTTCCCTGTATTGCTGAAATGTGATTTCTCTTGATTTCAACCTAT
CATAGGACGTTTCTATAAGATGCGTGTTTCTTGAGAATTTAACATTTACAACCTTTTTAAGTCCTTTTATT
AACACGGTGTTATCGTTTTCTAACACGATGTGAATATTATCTGTGGCTAGATAGTAAATATAATGTGAGAC
GTTGTGACGTTTTAGTTCAGAATAAAACAATTCACAGTCTAAATCTTTTCGCACTTGATCGAATATTTCTT
TAAAAATGGCAACCTGAGCCATTGGTAAAACCTTCCATGTGATACGAGGGCGCGTAGTTTGCATTATCGTT
TTTATCGTTTCAATCTGGTCTGACCTCCTTGTGTTTTGTTGATGATTTATGTCAAATATTAGGAATGTTTT
CACTTAATAGTATTGGTTGCGTAACAAAGTGCGGTCCTGCTGGCATTCTGGAGGGAAATACAACCGACAGA
TGTATGTAAGGCCAACGTGCTCAAATCTTCATACAGAAAGATTTGAAGTAATATTTTAACCGCTAGATGAA
GAGCAAGCGCATGGAGCGACAAAATGAATAAAGAACAATCTGCTGATGATCCCTCCGTGGATCTGATTCGT
GTAAAAAATATGCTTAATAGCACCATTTCTATGAGTTACCCTGATGTTGTAATTGCATGTATAGAACATAA
GGTGTCTCTGGAAGCATTCAGAGCAATTGAGGCAGCGTTGGTGAAGCACGATAATAATATGAAGGATTATT
CCCTGGTGGTTGACTGATCACCATAACTGCTAATCATTCAAACTATTTAGTCTGTGACAGAGCCAACACGC
AGTCTGTCACTGTCAGGAAAGTGGTAAAACTGCAACTCAATTACTGCAATGCCCTCGTAATTAAGTGAATT
TACAATATCGTCCTGTTCGGAGGGAAGAACGCGGGATGTTCATTCTTCATCACTTTTAATTGATGTATATG
CTCTCTTTTCTGACGTTAGTCTCCGACGGCAGGCTTCAATGACCCAGGCTGAGAAATTCCCGGACCCTTTT
TGCTCAAGAGCGATGTTAATTTGTTCAATCATTTGGTTAGGAAAGCGGATGTTGCGGGTTGTTGTTCTGCG
GGTTCTGTTCTTCGTTGACATGAGGTTGCCCCGTATTCAGTGTCGCTGATTTGTATTGTCTGAAGTTGTTT
TTACGTTAAGTTGATGCAGATCAATTAATACGATACCTGCGTCATAATTGATTATTTGACGTGGTTTGATG
GCCTCCACGCACGTTGTGATATGTAGATGATAATCATTATCACTTTACGGGTCCTTTCCGGTGATCCGACA
GGTTACGGCCTGATGCGGTATTTTCTCCTTACGCATCTGTGCGGTATTTCACACCGCATACGTCAAAGCAA
CCATAGTACGCGCCCTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGTTCGCCGGCTTTC
CCCGTCAAGCTCTAAATCGGGGGCTCCCTTTAGGGTTCCGATTTAGTGCTTTACGGCACCTCGACCCCAAA
AAACTTGATTTGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCTCGGGCTATT
CTTTTGATTTATAAGGGATTTTGCCGATTTCGGCCTATTGGTTAAAAAATGAGCTGATTTAACAAAAATTT
AACGCGAATTTTAACAAAATATTAACGTTTACAATTTTATGGTGCACTCTCAGTACAATCTGCTCTGATGC
CGCATAGTTAAGCCAGCCCCGACACCCGCCAACACCCGCTGACGCGCCCTGACGGGCTTGTCTGCTCCCGG
CATCCGCTTACAGACAAGCTGTGACCGTCTCCGGGAGCTGCATGTGTCAGAGGTTTTCACCGTCATCACCG
AAACGCGCGAGACGAAAGGGCCTCGTGATACGCCTATTTTTATAGGTTAATGTCATGATAATAATGGTTTC
TTAGACGTCAGGTGGCACTTTTCGGGGAAATGTGCGCGGAACCCCTATTTGTTTATTTTTCTAAATACATT
CAAATATGTATCCGCTCATGAGACAATAACCCTGATAAATGCTTCAATAATATTGAAAAAGGAAGAGTATG
AGCCATATTCAACGGGAAACGTCGAGGCCGCGATTAAATTCCAACATGGATGCTGATTTATATGGGTATAA
ATGGGCTCGCGATAATGTCGGGCAATCAGGTGCGACAATCTATCGCTTGTATGGGAAGCCCGATGCGCCAG
AGTTGTTTCTGAAACATGGCAAAGGTAGCGTTGCCAATGATGTTACAGATGAGATGGTCAGACTAAACTGG
CTGACGGAATTTATGCCTCTTCCGACCATCAAGCATTTTATCCGTACTCCTGATGATGCATGGTTACTCAC
CACTGCGATCCCCGGAAAAACAGCATTCCAGGTATTAGAAGAATATCCTGATTCAGGTGAAAATATTGTTG
ATGCGCTGGCAGTGTTCCTGCGCCGGTTGCATTCGATTCCTGTTTGTAATTGTCCTTTTAACAGCGATCGC
GTATTTCGTCTCGCTCAGGCGCAATCACGAATGAATAACGGTTTGGTTGATGCGAGTGATTTTGATGACGA
GCGTAATGGCTGGCCTGTTGAACAAGTCTGGAAAGAAATGCATAAACTTTTGCCATTCTCACCGGATTCAG
TCGTCACTCATGGTGATTTCTCACTTGATAACCTTATTTTTGACGAGGGGAAATTAATAGGTTGTATTGAT
GTTGGACGAGTCGGAATCGCAGACCGATACCAGGATCTTGCCATCCTATGGAACTGCCTCGGTGAGTTTTC
TCCTTCATTACAGAAACGGCTTTTTCAAAAATATGGTATTGATAATCCTGATATGAATAAATTGCAGTTTC
ATTTGATGCTCGATGAGTTTTTCTAACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCTCTGCTAATCCTGTTAC
CAGTGGCTGCTGCCAGTGGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTCACATGT
SEQ ID NO: 14 Forward primer for the qPCR targeting RBG polyA
GCCAAAAATTATGGGGACAT
SEQ ID NO: 15 Reverse primer for the qPCR targeting RBG polyA
ATTCCAACACACTATTGCAATG
SEQ ID NO: 16 Probe for the qPCR targeting RBG polyA
ATGAAGCCCCTTGAGCATCTGACTTCT
SEQ ID NO: 17 Forward primer specific for the murine beta-actin
CCTCTATGCCAACACAGTGC
SEQ ID NO: 18 Reverse primer specific for the murine beta-actin
GCTAGGAGCCAGAGCAGTAA
SEQ ID NO: 19 Probe specific for the murine beta-actin
ACCCAGGCATTGCTGACAGGATGCA
SEQ ID NO: 20 Forward primer specific for the hGLA gene
ACAGTACTGCAACCACTGGA
SEQ ID NO: 21 Reverse primer specific for the hGLA gene
GTCCAGTCCAGGATGCTCTT
SEQ ID NO: 22 Probe specific for the murine hGLA gene
CGCCGACATCGACGACAGCTGG
SEQ ID NO: 23 Forward primer specific for the murine GAPDH gene
AGTGTTTCCTCGTCCCGTAG
SEQ ID NO: 24 Reverse primer specific for the murine GAPDH gene
AATCTCCACTTTGCCACTGC
SEQ ID NO: 25 Probe specific for the murine GAPDH gene
TGGGCGCCTGGTCACCAGGG
SEQ ID NO: 26 stuffer sequence
CTGAATTATATACTCGGTGGATCGTATCTTGAAAATTCTTACTTTTATGTAACGTATAAAGATTAGTGAGA
AACAGTTCTTTCTATCCTCTCCTCTCTATTTTATCAACTACCCCACCTCTCCAGATATAAACTTACATCAG
ATTTTTAACAAGAGAATTCTAACCTTCATACATCCGACCCTCCCATTTATGGTTTAGAACCATATAGTCTT
GACTCGTACAGGGAACTTCCAATTCTTTATCAATTACCCGTTTATCTCGTACCGTTATAAAACATCTCGTC
GTTCATCATCCGGAACTTATCTACAGCGAGTTTTTCATTATACATTCGACTTGTGTTTTTACATTGTTTAC
TTAAATCTATGTATAAACTTATAATTTAAGTCCAACAAACCCTCTACGTGGATCAGAAACTACCAATTTGG
AAAGGGAGGTATCTTCTCTGTCTCTGTCTTACCGAACGACCTGATTACAGGGTTAAGTTATCTCAGAATAG
ATACTTCCAATTTTTGTTCTTCTCTGTATCACTCCTCATAAAGTAATGACGTACACAAACATCAGAACTAT
CGTTCTTGACAGTTAAGTTCGATCGTTGAAAAAGGACTTCATCACTAATATAAAGAATCTCCTTTCATAAC
CTCACAACGGGAATAATACGACTATTCTCATGGGTCTTATTTTACCTTTTTCTACTTGTTAATTGACTATT
TAAACACGTGTACCGTTTAATCAATTACCCTTGGTATCCTCTTAAATAAAGATCTACATTTATTAATAAAA
TTCAAACGGGATACCACCGGGGTGTGTACTCTGTTTGGGGGTTCTACACTGAAAACTCTTACTCTGAACCT
ATTTTTTGTACATCTTTACGTTCGGGACTTCGAGTTGAGGGATAACGATAGTGTCCCCAATATTAACGTAT
TTTAAATCGATATCTTTCAACGACAGTAGAGAACACCCGACATTAGTAGCAGATCCGAATTCTCATTATAA
CGTTTTGGACAGTACGGGTGTGTTTAGAGAGGGACCGTAACAACAGAAACGTCTACAGTCACTTTCTCTTG
GTCGTCGAGGGTACTCAAAC
SEQ ID NO: 27 stuffer sequence
ATCTCGTCTCTTTCTTCCTCTATTTCTATGTCTGATGTCGGATAAGTCTCACTGAATGTTTACGTGGCAAG
ACCTATCCTACGGCACCCCGTCGGAGAGTCGTGTGTATAGTCGATCCGATTAATATACAATTAACATCAAT
AAATAGGACCAAAATATTATATTAAAGAGACCTTCAACAATCTTAAAAATAAGATTGAAATTACAAGACTT
TAAAGTATATGTATACCTTATTTAACGAAACCTAAAAATAAACAAAATAAAAGGTAAACTAAATTAAACCA
AAACATTTAGGACGAGTTGTGACTCATCCCAGAAAAAAGATTAGAGGATTGAAATAAAGAGATATTTCCTT
ATAAGAGACAATAGAGAATTTTATAACCGTCGAGGCATAATGCTGATTAGAAAACTTTGAGATAAATCTAC
TAAAAACCCTTGAAGACCCAGACAAGAGGTTAAGAGAAGGGAAAATAATAAAGAGACAGAAATCAAAGTCG
AAATATTAATTAAGATGACGGTGTATGTAAAACGTGAAACCGGATATTTAAAGTGTTAAACACAAAAAAAT
AAAAATATCAAGATCAACCAAGAAGAAATATAGGTGGATCAGTACAAACGGATGTGTGTTCAGTGTTTCTA
AAAGAGGATATAAAAAATAGATTTTCGAAATATCAAAACAAAAATATAAAGTCAGATATTATGTAGAGTTT
AATTAAAAACACAAAACACACTTCATCCCAGATCCATATAACGAAAGGGTTTCCTGTCGATCAAAAGGATT
GTGCTAAACAATTCTAATAAGTAAAAGGATTACTTAAAAACTGGTTTGTTTAGTTTACCTGTAGATTCACA
CAGAGATCAAAAATCGGGAAATAAGATAATGAAATTAGATAAATAACCAAGACACGGTCATTTTATTATTA
AACTAATGTCGTATAATATCAATTAGAACTCCAATACATCACATACAGAGGGTTCAAACAAGTAGAAAGAG
TTCAAACGAAACCTATCAGATCCAGGAATTCCATGGAAATGAGTCCTCGAAAACTTGTGTCATCGTAAGAT
CGTCTTTACTCGGAACTGTT

1. A recombinant adeno-associated virus (rAAV) comprising a genome comprising an expression construct comprising a polynucleotide of interest that comprises a nucleotide sequence selected from SEQ ID NOs: 1, 2, 3 and 4, and is operably linked to a promoter.

2. The rAAV of claim 1, wherein the construct further comprises an intron.

3. The rAAV of claim 2, wherein the intron is between the promoter and the polynucleotide of interest.

4. The rAAV of claim 2, wherein the intron is at least 200 nucleotides in length.

5. The rAAV claim 2, wherein the intron is selected from a chicken beta-actin (CBA) chimeric intron and a modified CBA chimeric intron set forth in SEQ ID NOs: 5 and 6, respectively.

6. The rAAV of claim 1, wherein the construct further comprises an enhancer.

7. The rAAV of claim 6, wherein the enhancer is upstream of the promoter.

8. The rAAV of claim 6, wherein the enhancer is a CMV early enhancer set forth in SEQ ID NO: 9.

9. The rAAV of claim 1, wherein the promoter is a chicken beta-actin core promoter set forth in SEQ ID NO: 10.

10. A pharmaceutical composition comprising the rAAV of claim 1.

11. The rAAV or the pharmaceutical composition of claim 10, for use in the treatment of a disease associated with the deficiency of alpha galactosidase A (α-Gal A).

12. The rAAV or the pharmaceutical composition of claim 10, wherein the disease is Fabry disease.


SEQUENCE LISTING (27 sequences)

SEQ ID NO: 1 - DNA, 1293 bp, synthetic construct
  source 1..1293: mol_type = other DNA
atgcagctgagaaatcctgagctgcacctgggctgtgccctggctctgagatttctggctctggtgtcctgggacatccctggcgctagagccctggataacggcctggccagaacacctacaatgggctggctgcactgggagagattcatgtgcaacctggactgccaagaggaacccgacagctgcatcagcgagaagctgttcatggaaatggccgagctgatggtgtccgaaggctggaaggacgccggctacgagtacctgtgcatcgacgactgttggatggcccctcagagagactctgagggcagactgcaggccgatcctcagagatttccccacggcattagacagctggccaactacgtgcacagcaagggcctgaagctgggcatctacgccgacgtgggcaacaagacctgtgccggctttcctggcagcttcggctactacgatatcgacgcccagaccttcgccgattggggagtcgatctgctgaagttcgacggctgctactgcgacagcctggaaaatctggccgacggctacaagcacatgtctctggccctgaatcggaccggcagatccatcgtgtacagctgcgagtggcccctgtacatgtggcccttccagaagcctaactacaccgagatcagacagtactgcaaccactggcggaacttcgccgacatcgacgatagctggaagtccatcaagagcatcctggactggaccagcttcaatcaagagcggatcgtggacgtggcaggacctggcggatggaacgatcctgacatgctggtcatcggcaacttcggcctgagctggaaccagcaagtgacccagatggccctgtgggccattatggccgctcctctgttcatgagcaacgacctgagacacatcagccctcaggccaaggctctgctgcaggacaaggatgtgatcgctatcaaccaggatcctctgggcaagcagggctaccagctgagacagggcgacaatttcgaagtgtgggaaagacccctgagcggactggcttgggccgtcgccatgatcaacagacaagagatcggcggaccccggtcctacacaattgccgtggcttctctcggcaaaggcgtggcctgtaatcccgcctgctttatcacacagctgctgcccgtgaagagaaagctgggcttttacgagtggaccagcagactgcggagccacatcaatcctaccggcacagtgctgctgcagctggaaaacacaatgcagatgagcctgaaggacctgctgtgatga

SEQ ID NO: 2 - DNA, 1293 bp, synthetic construct
  source 1..1293: mol_type = other DNA
atgcagctccgaaacccggaattgcatttgggctgtgcgctggcgctcagatttctggcactcgtctcttgggacattcctggggcgcgggcgctcgacaacggcctggcacgaaccccaacgatgggctggttgcattgggagcggtttatgtgtaaccttgactgtcaagaggaaccggatagctgtatcagtgagaagctgtttatggaaatggcagaattgatggtgtccgaagggtggaaagacgcgggatacgagtacctctgcatcgacgactgctggatggcaccccagcgggactcagaagggaggctgcaggctgaccctcaacggtttccccacgggataagacaactggcgaactacgttcattccaaaggactcaaactgggtatctacgctgacgtcggaaataagacgtgtgcgggctttccagggagcttcgggtactacgacatcgacgcccaaacgtttgcggactggggcgttgacttgctcaaatttgacggctgctactgtgacagcctggaaaatctggcagatggctataaacatatgagtctcgcccttaaccggactggtagatcaattgtatattcctgcgagtggcctttgtacatgtggccattccagaaaccgaactatactgagatacgccaatattgcaatcattggcgaaatttcgccgacatagatgatagttggaagtctataaagtctattcttgattggacttcttttaaccaggagcgcattgtggacgttgcggggcccggagggtggaacgatcccgatatgcttgtgatcggcaacttcggtctgtcctggaaccaacaagtcacccaaatggcattgtgggcgataatggctgcgcccctgttcatgtctaatgatctcaggcacataagcccccaggctaaagcgcttcttcaagataaggacgttatagctatcaaccaggacccccttgggaagcagggctaccagcttcgacaaggagataatttcgaggtgtgggagcgccccctgtccgggcttgcgtgggcggtcgccatgatcaaccgacaggaaattgggggccccagatcatatacgatcgcagttgctagcctcggaaaaggcgttgcttgcaatcccgcttgcttcattacgcaactcctccccgttaagcggaaactgggattctacgagtggacatcccggctgcgatcacacataaatcccactgggacagtcctcctccagctggagaatacaatgcaaatgagccttaaagacctcttgtgatga

SEQ ID NO: 3 - DNA, 1293 bp, synthetic construct
  source 1..1293: mol_type = other DNA
atgcagctgaggaacccagagctgcacctgggctgcgcactggccctgagattcctggccctggtgtcctgggacatccctggagcacgggccctggataacggactggcaagaaccccaacaatgggctggctgcactgggagcggttcatgtgcaatctggactgtcaggaggagccagatagctgtatctccgagaagctgtttatggagatggccgagctgatggtgtctgagggctggaaggacgccggctacgagtatctgtgcatcgacgattgttggatggcaccacagagggacagcgagggcagactgcaggccgatccccagaggtttcctcacggcatccgccagctggccaactacgtgcacagcaagggcctgaagctgggcatctacgccgacgtgggcaataagacctgcgccggcttccctggctcctttggctactacgacatcgacgcccagacattcgccgactggggcgtggatctgctgaagtttgacggctgctactgtgattccctggagaacctggccgacggctataagcacatgtctctggccctgaataggaccggccgctctatcgtgtacagctgcgagtggccactgtatatgtggccattccagaagcccaactacacagagatccggcagtattgtaaccactggagaaattttgccgacatcgacgattcttggaagagcatcaagtccatcctggactggacctccttcaaccaggagaggatcgtggacgtggcaggacctggaggctggaacgacccagatatgctggtcatcggcaacttcggcctgtcttggaatcagcaggtgacacagatggcactgtgggcaatcatggcagcaccactgtttatgagcaatgatctgaggcacatctccccacaggcaaaggccctgctgcaggacaaggatgtgatcgccatcaaccaggaccccctgggcaagcagggctaccagctgaggcagggcgataattttgaagtgtgggagaggccactgtccggactggcctgggccgtggccatgatcaacaggcaggagatcggcggccctcgctcttacaccatcgcagtggcaagcctgggcaagggagtggcatgcaatccagcatgtttcatcacccagctgctgcctgtgaagcggaagctgggcttttatgagtggacatcccggctgagatctcacatcaaccctaccggcacagtgctgctgcagctggagaatacaatgcagatgagcctgaaggacctgctgtgatga

SEQ ID NO: 4 - DNA, 1293 bp, synthetic construct
  source 1..1293: mol_type = other DNA
atgcagctgagaaaccccgagctgcacctgggctgcgccctggccctgagattcctggccctggtgagctgggacatccccggcgccagagccctggacaacggcctggccagaacccccaccatgggctggctgcactgggagagattcatgtgcaacctggactgccaggaggagcccgacagctgcatcagcgagaagctgttcatggagatggccgagctgatggtgagcgagggctggaaggacgccggctacgagtacctgtgcatcgacgactgctggatggccccccagagagacagcgagggcagactgcaggccgacccccagagattcccccacggcatcagacagctggccaactacgtgcacagcaagggcctgaagctgggcatctacgccgacgtgggcaacaagacctgcgccggcttccccggcagcttcggctactacgacatcgacgcccagaccttcgccgactggggcgtggacctgctgaagttcgacggctgctactgcgacagcctggagaacctggccgacggctacaagcacatgagcctggccctgaacagaaccggcagaagcatcgtgtacagctgcgagtggcccctgtacatgtggcccttccagaagccgaactacaccgagatcagacagtactgcaaccactggagaaacttcgccgacatcgacgacagctggaagagcatcaagagcatcctggactggaccagcttcaaccaggagagaatcgtggacgtggccggccccggcggctggaacgaccccgacatgctggtgatcggcaacttcggcctgagctggaaccagcaggtgacccagatggccctgtgggccatcatggccgcccccctgttcatgagcaacgacctgagacacatcagcccccaggccaaggccctgctgcaggacaaggacgtgatcgccatcaaccaggaccccctgggcaagcagggctaccagctgagacagggcgacaacttcgaggtgtgggagagacccctgagcggcctggcctgggccgtggccatgatcaacagacaggagatcggcggccccagaagctacaccatcgccgtggccagcctgggcaagggcgtggcctgcaaccccgcctgcttcatcacccagctgctgcccgtgaagagaaagctgggcttctacgagtggaccagcagactgagaagtcacatcaaccccaccggcaccgtgctgctgcagctggagaacaccatgcagatgagcctgaaggacctgctgtgatga

SEQ ID NO: 5 - DNA, 1065 bp, synthetic construct
  source 1..1065: mol_type = other DNA
gagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 6 - DNA, 403 bp, synthetic construct
  source 1..403: mol_type = other DNA
gagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 7 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 8 - DNA, 157 bp, synthetic construct
  source 1..157: mol_type = other DNA
aactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagct

SEQ ID NO: 9 - DNA, 382 bp, synthetic construct
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 10 - DNA, 282 bp, synthetic construct
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 11 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 12 - protein, 429 aa, synthetic construct
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 13 - DNA, 9863 bp, synthetic construct
  source 1..9863: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcaattgctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggaagcttgagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggcctcgaggacggggtgaactacgcctgaggatccgccaccatgcagctgagaaaccccgagctgcacctgggctgcgccctggccctgagattcctggccctggtgagctgggacatccccggcgccagagccctggacaacggcctggccagaacccccaccatgggctggctgcactgggagagattcatgtgcaacctggactgccaggaggagcccgacagctgcatcagcgagaagctgttcatggagatggccgagctgatggtgagcgagggctggaaggacgccggctacgagtacctgtgcatcgacgactgctggatggccccccagagagacagcgagggcagactgcaggccgacccccagagattcccccacggcatcagacagctggccaactacgtgcacagcaagggcctgaagctgggcatctacgccgacgtgggcaacaagacctgcgccggcttccccggcagcttcggctactacgacatcgacgcccagaccttcgccgactggggcgtggacctgctgaagttcgacggctgctactgcgacagcctggagaacctggccgacggctacaagcacatgagcctggccctgaacagaaccggcagaagcatcgtgtacagctgcgagtggcccctgtacatgtggcccttccagaagccgaactacaccgagatcagacagtactgcaaccactggagaaacttcgccgacatcgacgacagctggaagagcatcaagagcatcctggactggaccagcttcaaccaggagagaatcgtggacgtggccggccccggcggctggaacgaccccgacatgctggtgatcggcaacttcggcctgagctggaaccagcaggtgacccagatggccctgtgggccatcatggccgcccccctgttcatgagcaacgacctgagacacatcagcccccaggccaaggccctgctgcaggacaaggacgtgatcgccatcaaccaggaccccctgggcaagcagggctaccagctgagacagggcgacaacttcgaggtgtgggagagacccctgagcggcctggcctgggccgtggccatgatcaacagacaggagatcggcggccccagaagctacaccatcgccgtggccagcctgggcaagggcgtggcctgcaaccccgcctgcttcatcacccagctgctgcccgtgaagagaaagctgggcttctacgagtggaccagcagactgagaagtcacatcaaccccaccggcaccgtgctgctgcagctggagaacaccatgcagatgagcctgaaggacctgctgtgatgatgtacaggtacctcgatggatttccaaggttaattcattggaattgaaaattaacagagatctagagctgaattcctgcagccagggggatcagcctgatctttttccctctgccaaaaattatggggacatcatgaagccccttgagcatctgacttctggctaataaaggaaatttattttcattgcaatagtgtgttggaattttttgtgtctctcactcgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 20 bp, synthetic construct
  source 1..20: mol_type = other DNA
gccaaaaattatggggacat

SEQ ID NO: 15 - DNA, 22 bp, synthetic construct
  source 1..22: mol_type = other DNA
attccaacacactattgcaatg

SEQ ID NO: 16 - DNA, 27 bp, synthetic construct
  source 1..27: mol_type = other DNA
atgaagccccttgagcatctgacttct

SEQ ID NO: 17 - DNA, 20 bp, synthetic construct
  source 1..20: mol_type = other DNA
cctctatgccaacacagtgc

SEQ ID NO: 18 - DNA, 20 bp, synthetic construct
  source 1..20: mol_type = other DNA
gctaggagccagagcagtaa

SEQ ID NO: 19 - DNA, 25 bp, synthetic construct
  source 1..25: mol_type = other DNA
acccaggcattgctgacaggatgca

SEQ ID NO: 20 - DNA, 20 bp, synthetic construct
  source 1..20: mol_type = other DNA
acagtactgcaaccactgga

SEQ ID NO: 21 - DNA, 20 bp, synthetic construct
  source 1..20: mol_type = other DNA
gtccagtccaggatgctctt

SEQ ID NO: 22 - DNA, 22 bp, synthetic construct
  source 1..22: mol_type = other DNA
cgccgacatcgacgacagctgg

SEQ ID NO: 23 - DNA, 20 bp, synthetic construct
  source 1..20: mol_type = other DNA
agtgtttcctcgtcccgtag

SEQ ID NO: 24 - DNA, 20 bp, synthetic construct
  source 1..20: mol_type = other DNA
aatctccactttgccactgc

SEQ ID NO: 25 - DNA, 20 bp, synthetic construct
  source 1..20: mol_type = other DNA
tgggcgcctggtcaccaggg

SEQ ID NO: 26 - DNA, 1085 bp, synthetic construct
  source 1..1085: mol_type = other DNA
ctgaattatatactcggtggatcgtatcttgaaaattcttacttttatgtaacgtataaagattagtgagaaacagttctttctatcctctcctctctattttatcaactaccccacctctccagatataaacttacatcagatttttaacaagagaattctaaccttcatacatccgaccctcccatttatggtttagaaccatatagtcttgactcgtacagggaacttccaattctttatcaattacccgtttatctcgtaccgttataaaacatctcgtcgttcatcatccggaacttatctacagcgagtttttcattatacattcgacttgtgtttttacattgtttacttaaatctatgtataaacttataatttaagtccaacaaaccctctacgtggatcagaaactaccaatttggaaagggaggtatcttctctgtctctgtcttaccgaacgacctgattacagggttaagttatctcagaatagatacttccaatttttgttcttctctgtatcactcctcataaagtaatgacgtacacaaacatcagaactatcgttcttgacagttaagttcgatcgttgaaaaaggacttcatcactaatataaagaatctcctttcataacctcacaacgggaataatacgactattctcatgggtcttattttacctttttctacttgttaattgactatttaaacacgtgtaccgtttaatcaattacccttggtatcctcttaaataaagatctacatttattaataaaattcaaacgggataccaccggggtgtgtactctgtttgggggttctacactgaaaactcttactctgaacctattttttgtacatctttacgttcgggacttcgagttgagggataacgatagtgtccccaatattaacgtattttaaatcgatatctttcaacgacagtagagaacacccgacattagtagcagatccgaattctcattataacgttttggacagtacgggtgtgtttagagagggaccgtaacaacagaaacgtctacagtcactttctcttggtcgtcgagggtactcaaac

SEQ ID NO: 27 - DNA, 1085 bp, synthetic construct
  source 1..1085: mol_type = other DNA
atctcgtctctttcttcctctatttctatgtctgatgtcggataagtctcactgaatgtttacgtggcaagacctatcctacggcaccccgtcggagagtcgtgtgtatagtcgatccgattaatatacaattaacatcaataaataggaccaaaatattatattaaagagaccttcaacaatcttaaaaataagattgaaattacaagactttaaagtatatgtataccttatttaacgaaacctaaaaataaacaaaataaaaggtaaactaaattaaaccaaaacatttaggacgagttgtgactcatcccagaaaaaagattagaggattgaaataaagagatatttccttataagagacaatagagaattttataaccgtcgaggcataatgctgattagaaaactttgagataaatctactaaaaacccttgaagacccagacaagaggttaagagaagggaaaataataaagagacagaaatcaaagtcgaaatattaattaagatgacggtgtatgtaaaacgtgaaaccggatatttaaagtgttaaacacaaaaaaataaaaatatcaagatcaaccaagaagaaatataggtggatcagtacaaacggatgtgtgttcagtgtttctaaaagaggatataaaaaatagattttcgaaatatcaaaacaaaaatataaagtcagatattatgtagagtttaattaaaaacacaaaacacacttcatcccagatccatataacgaaagggtttcctgtcgatcaaaaggattgtgctaaacaattctaataagtaaaaggattacttaaaaactggtttgtttagtttacctgtagattcacacagagatcaaaaatcgggaaataagataatgaaattagataaataaccaagacacggtcattttattattaaactaatgtcgtataatatcaattagaactccaatacatcacatacagagggttcaaacaagtagaaagagttcaaacgaaacctatcagatccaggaattccatggaaatgagtcctcgaaaacttgtgtcatcgtaagatcgtctttactcggaactgtt